(19) United States
(12) Patent Application Publication
(10) Pub. No.: US 2021/0171581 A1
(43) Pub. Date: Jun. 10, 2021

(54) IMMUNE ACTIVATION TRIGGERED BY FILOVIRUS PROTEINS AND POLYPEPTIDES

(71) Applicant: University of Hawaii, Honolulu, HI (US)

(72) Inventors: Axel Lehrer, Honolulu, HI (US); Saguna Verma, Honolulu, HI (US)

(21) Appl. No.: 16/613,742

(22) PCT Filed: May 15, 2018
(86) PCT No.: PCT/US2018/032751

§ 371 Date: Nov. 14, 2019

Related U.S. Application Data
(60) Provisional application No. 62/506,557, filed on May 15, 2017.

(51) Int. Cl. C07K 14/005; C12N 7/00; A61K 39/12; A61P 31/14

(52) U.S. Cl. CPC C07K 14/005; C12N 7/00; A61K 39/12; A61P 31/14; A61K 2039/70; C12N 2760/14034; C12N 2760/14122; C12N 2760/14134; C12N 2760/14022

(57) ABSTRACT

Provided herein are compositions that include an isolated glycosylated polypeptide comprising at least 50 amino acid residues of a surface glycoprotein of a filovirus and a pharmaceutically acceptable carrier. The glycosylated polypeptide corresponds to one or more structural subunits of the glycoprotein. When the compositions are administered to a subject, they stimulate an innate immune response. The response can be stimulated with administration in the absence of an adjuvant. Methods of using the compositions and for their production are also disclosed.

### CROSS-REFERENCE TO RELATED APPLICATION

This application claims the benefit under 35 U.S.C. § 119(e) of U.S. Ser. No. 62/506,557, filed May 15, 2017, the entire content of which is incorporated herein in its entirety by reference.

### STATEMENT REGARDING FEDERALLY SPONSORED RESEARCH OR DEVELOPMENT

This invention was made with government support under Grant Nos. RO1 AI119185 and P30 GM114737 awarded by National Institutes of Health. The government has certain rights in the invention.

### INCORPORATION OF SEQUENCE LISTING

The material in the accompanying sequence listing is hereby incorporated by reference into this application. The accompanying sequence listing text file, name HBI160_1WO_Sequence_Listing.txt, was created on May 14, 2018, and is 10 kb. The file can be accessed using Microsoft Word on a computer that uses Windows OS.

### FIELD OF THE INVENTION

The present invention relates generally to immune activation, and more specifically to filovirus antigens for inducing an innate immune response.

### BACKGROUND INFORMATION

Ebola virus (EBOV), a member of the Filoviridae family, causes the most severe form of viral hemorrhagic fever. The recent outbreak of Ebola virus (EBOV, also known as Zaire ebolavirus) in several West African countries in 2013-16 is by far the largest and most complex filovirus outbreak and has brought the virus and Ebola virus disease (EVD) to the forefront of interest as an emerging infectious disease. The World Health Organization has reported 28,616 confirmed and suspected cases and 11,310 deaths. The quick spread of EBOV infection outside the outbreak regions into other African countries such as Nigeria and Mali, and the United States indicates that EBOV has become a global threat to public health and uncertainty exists regarding future outbreaks of EBOV and other filoviruses such as Marburg virus (MARV).

EBOV is a member of the Filoviridae family and is classified in the genus Ebolavirus, species Zaire ebolavirus. It causes severe disease and high case fatality rates in humans. The single stranded, negative-sense RNA genome of EBOV encodes seven viral structural proteins including nucleoprotein (NP), and virion protein (VP) 35, VP40, glycoprotein (GP), VP30, VP24, and RNA-dependent RNA polymerase (L). The open reading frame (ORF) coding for EBOV GP also gives rise to non-structural soluble GP (sGP) and shed GP, which is generated from the mature trimeric surface GP via proteolytic cleavage of the transmembrane region by TACE (TNF-α converting enzyme), and released from infected cells.

There is currently no FDA approved antiviral therapy or vaccine available for prevention of EVD and treatment is limited to supportive care. While cocktails of monoclonal antibodies as well as antivirals have been tested as experimental therapies mainly during the recent West African outbreak, a safe and efficacious vaccine is still the most economic and effective countermeasure to prevent large-scale filovirus outbreaks. Several vaccine approaches including virally-vectored vaccines such as recombinant vesicular stomatitis virus (rVSV), recombinant adenoviruses, and protein-based subunit vaccines such as virus-like particles (VLPs) have been demonstrated to protect against filovirus infection in both small animal models such as mice as well as in non-human primates (NHPs). However, despite active research on EBOV vaccines, the specific mechanisms by which GP mediates immune protection are not yet fully understood.

Among the many problems currently faced are insufficient safety of potential vaccines, low expression yields of antigens, insufficient efficacy of potential vaccines and a concomitant need for using adjuvants, low durability of potential antigens, and non-immunogenic conformations of potential antigens. Furthermore, because Ebola virus has to be handled under maximum level biocontainment, development of proper antigens is laborious and slow.

Thus, there is a need for improved immune activation, for faster immune activation, and for broader immune protection against different types of filoviruses.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention is based, in part, on the development of filovirus antigens. In particular, recombinant protein subunit antigens are being disclosed. The subunit antigens enable, individually or in combination, generation of an innate immune response even when no adjuvant is used. This is in contrast to the traditional vaccine methodology, which mainly is concerned with an adaptive response and which routinely depends on use of adjuvants.

Significance of the present disclosure is apparent from the lack of an FDA approved vaccine to prevent infection by the highly pathogenic Ebola virus.

Zaire ebolavirus (EBOV) is the most virulent member in the filovirus family that causes severe viral hemorrhagic fevers with a mortality of up to 90% in humans and non-human primates (NHPs). The 2013-2016 EBOV outbreak in West Africa with more than 28,000 cases and 11,000 deaths highlighted the potential of filovirus infections as a global public health threat. No FDA approved antivirals or vaccines are available to prevent or control future filovirus outbreaks. While antibody therapy such as passive transfer of polyclonal IgG or cocktails of monoclonal antibodies was shown to protect NHPs from EBOV infections and also has been used in human clinical trials (with inconclusive results), a safe and efficacious vaccine is still the most economic and effective intervention for large-scale filovirus outbreaks. Several Ebola vaccine candidates have entered clinical trials, including virally vectored vaccines using recombinant vesicular stomatitis virus (rVSV) or adenoviral vectors and protein-based subunit vaccines such as virus-like particles (VLPs). However, the correlates of protection in humans and animal models are not identified yet. Although the results of human clinical trials using the furthest progressed vaccine candidate rVSV-ZEBOV, in clinical development led by Merck, demonstrated its safety and efficacy, concerns regarding rapid decline of IgM antibody and about undesirable reactogenicity have been raised in 3 separate phase I clinical trials. Moreover, the second immunization did not have a boost effect due to the attenuating effect of pre-existing antibody on rVSV replication. This explains why there is still an urgent need to understand the mechanisms of immunogenicity and immune protection in detail to develop safe and effective EBOV vaccines.

The recombinant subunit platform described here offers a safe, non-replicating alternative to other vaccine candidates and a tool to study mechanism of action of vaccine antigen without other complex components introduced by virally-vectored vaccines. We have developed a recombinant protein-based subunit vaccine using a platform in which antigen is expressed from stably transformed Drosophila S2 cells and have demonstrated its immunogenicity and efficacy in rodents and NHPs. Considering potential use in children, pregnant women, and immunocompromised individuals in whom vaccination may trigger severe adverse effects, a subunit vaccine has a safety advantage over virally vectored vaccines which can furthermore be used in a prime-boost vaccination scheme. However, equally important, these highly purified recombinant proteins provide a unique tool for safely dissecting the protective immune response to a specific vaccine immunogen or a subdomain within the main antigen to also test different post-prophylactic immunization approaches.

Ebola virus glycoprotein (GP), as an antigen, can induce virus-neutralizing antibodies. However, the mechanisms by which GP confers protection remain unclear. We show that GP-induced innate immunity may limit EBOV infection and that an adaptive response is not the only way in which EBOV GP can confer protection. So far, no studies have extensively characterized the early immune responses to GP and how they control adaptive immunity. We describe rational antigen design that includes protective GP domains while minimizing undesirable cytopathic effects to provide optimal safety and protective efficacy for our subunit vaccine. The uncovered GP-mediated mechanisms of immune protection in this disclosure may also be observed in other platforms of EBOV vaccines such as rVSV-ZEBOV. The results from this disclosure provide a blueprint for future development of other EBOV and filovirus vaccines with a specific focus on rapid immune activation.

In an embodiment, disclosed is a composition including an isolated glycosylated polypeptide having at least about 20, 30, 40, 50 amino acid residues of the surface glycoprotein of a filovirus, wherein the glycosylated polypeptide corresponds to one or more structural subunits of the glycoprotein; and a pharmaceutically acceptable carrier.

In an aspect, the composition stimulates an innate immune response in a subject when it is administered to the subject. In various aspects, the composition stimulates the innate immune response when it is administered in the absence of an adjuvant. The administration of the composition to the subject may stimulate the innate immune response via the TLR4 pathway. In particular, the administration of the composition to the subject can stimulate production of one or more cytokines such as IL-1beta, IL-2, IL-4, IL-5, IL-6, IL-10, IFN-gamma, TNF-alpha, or combinations of such cytokines. In various aspects, the polypeptide may have 20-30 amino acid residues of the surface glycoprotein of a filovirus.

In some aspects of the composition, the glycosylated polypeptide includes at least 50 amino acid residues of the surface glycoprotein of a filovirus, which may be derived from Zaire ebolavirus (EBOV), Marburg marburgvirus (MARV) or a Sudan ebolavirus (SUDV). In various aspects, the glycosylated polypeptide has an amino acid sequence that is at least 95% identical to an amino acid sequence range among 1-501, 33-501, 33-201, 201-309, 309-501, and 502-676 of SEQ ID NO: 2. As an example, the glycosylated polypeptide has in an aspect an amino acid sequence that is at least 95% identical to amino acid sequence range 1-501 of SEQ ID NO: 2. The glycosylated polypeptide may include at least one N-linked glycosylation. In some aspects, the glycosylated polypeptide is only N-linked glycosylated, and is not O-linked glycosylated. In various aspects, the level of N-linked glycosylation of the glycosylated polypeptide is 10%, 20%, 30%, 40%, 50%, 60%, 70%, 80%, 90%, or 100% of the level of N-linked glycosylation of the corresponding subunit of the full length glycoprotein. Percentage identify of two amino acid sequences of unequal residue length is calculated with respect to the shorter of the two sequences.

In some aspects, the composition further includes one or more additional isolated glycosylated polypeptides each of which has at least 50 amino acid residues of the envelope glycoprotein of a filovirus, wherein each of the additional glycosylated polypeptides corresponds to one or more structural subunits of the glycoprotein. The composition can be a multivalent (e.g., bivalent, trivalent, tetravalent) formulation.

In some embodiments, disclosed is a method of inducing an innate immune response in a subject including administering an effective amount of the composition to the subject, thereby inducing an innate immune response. In some aspects, the effective amount of the composition is administered to the subject in absence of an adjuvant. The innate immune response thus induced can include production of cytokines, for example via a TLR4 pathway, or by activating other innate immune pathways. The induced innate immune response can enhance expression of costimulatory molecules CD40, CD80, and CD86 on surfaces of bone marrow-derived dendritic cells.

In various embodiments, disclosed are methods of producing the composition including expressing an antigen including the glycosylated polypeptide in Drosophila S2 cells and isolating the polypeptide. The method can further include purifying the glycosylated polypeptide using single-step immunoaffinity chromatography (IAC). The IAC can include an affinity column containing a monoclonal antibody. In some aspects, the purified glycosylated polypeptide has a three-dimensional structure that differs from the corresponding one or more structural subunits of the glycoprotein by less than 10 Angstroms in root-mean-square deviation of C alpha atomic coordinates after optimal rigid body superposition.

In many aspects, the isolated glycosylated polypeptide can be a recombinant one. Glycosylation of the polypeptide may be partial or in an equivalent amount to the corresponding native glycoprotein portions. The glycoprotein, in some embodiments is the surface glycoprotein of Ebola virus, Mayinga strain. The sequence of the glycoprotein, in some aspects, is obtained from Genbank accession number NC_002549. In various aspects, the correspondence between the polypeptide and the glycoprotein subunit(s) is with respect to the primary structure of the glycoprotein. In other aspects, the correspondence is in terms of the tertiary structure. In some embodiments, the polypeptide acts as both an immunogen and as an adjuvant. In some embodiments having multivalent antigens, the antigens come from different organisms, whereas in other embodiments, they are sourced from the same organism. In many embodiments, when innate immune response is activated, this can further lead to the activation of an adaptive immune response.

The embodiments described above have various advantages. For example, they allow mounting a rapid immune response. In addition, they allow different timing options for using the compositions. The immunogenic compositions can be used before an exposure, as a typical vaccine would be used, or they can be used post-exposure. Furthermore, compositions can confer broad immunity against multiple filoviruses, even when an antigen from only one source organism is used. The compositions can also confer protection against other infectious agents, as well as non-infectious conditions such as cancer. This is achieved by the immunomodulatory effects of the used compositions, which are in part linked to their effects on the innate immune response. By not needing an adjuvant, the compositions also both decrease the cost/speed of production and decrease risk of contamination. Moreover, when subunit antigens are used, because they do not replicate, safety is improved. The ability to use GP1 (N-terminal portion of GP truncated at amino acid 501 and separated from C-terminal portion after reduction of the intermolecular disulfide bond constituting amino acids 33-501), in an embodiment, allows a more robust innate immune response as compared to that from full-length surface GP (see, e.g., FIG. 18, panel A).

Other aspects and advantages of the invention will be apparent from the following description and the appended claims.

### BRIEF DESCRIPTION OF THE DRAWINGS

The foregoing and other objects of the present disclosure, the various features thereof, as well as the disclosure itself may be more fully understood from the following description, when read together with the accompanying drawings in which:

FIGS. 1A and 1B show that highly purified, recombinant EBOV GP induces potent antibody responses after two and three doses. (A) EBOV GP protein on SDS-PAGE stained using Coomassie blue. M, molecular weight marker, Lanes 1-2, 1 and 2 μg of single-step immunoaffinity purified GP protein (90% purity) and lanes 3-4, 1 and 2 μg of protein after 2-step purification (95% purity). (B) BALB/c mice were inoculated with GP (10 μg per dose) via subcutaneous route with or without adjuvant, followed by two booster doses in 4-week intervals. EBOV specific IgG was measured using a standard ELISA reporting endpoint titers (absorption >0.2 above background). Y-axis: GMT+95%CI;

FIGS. 2A-2E show that mouse bone marrow-derived macrophages (BMDMs) can efficiently internalize EBOV GP. BMDMs prepared from C57BL/6 mice were incubated with or without FITC-conjugated EBOV GP (10 μg/mL) at 37° C. for 30 min. The cells were washed and fixed, and the antigen uptake was evaluated by flow cytometry. (A-C) Live cells were gated based on FSC and SSC, and the percentage of FITC⁺cells was measured in untreated and GP-treated BMDMs. (D) Single parameter histogram of BMDMs incubated with and without GP-FITC. (E) Percentage of FITC⁺ BMDMs is expressed as mean±standard error of mean (SEM) of three independent experiments (C=medium control; GP=GP-FITC);

FIGS. 3A-3C show that EBOV GP induces gene expression of pro-inflammatory cytokines in mouse and human immune cells. BMDMs prepared from (A) BALB/c mice, (B) C57BL/6 mice, or (C) human THP-1 cells were treated with 1 μg/mL EBOV GP, and total cellular RNA was extracted. The change in the mRNA levels of TNF-α, IL-1β, and IL-6 at 2 and 6 h after treatment was analyzed by qRT-PCR using specific primers. The data was normalized to GAPDH mRNA and fold-change was calculated. The results are presented as mean±SEM of at least two independent treatments analyzed in duplicate wells;

FIGS. 4A and 4B show that EBOV GP stimulates the production of innate immune cytokines in mice. (FIG. 4A) BALB/c and (FIG. 4B) C57BL/6 mice were administered EBOV GP (100 μg/mouse) via the i.p. route. Mouse serum was collected at 6 and 24 h after administration. Levels of cytokines TNF-α, IL-1β, IL-6, IL-2, IL-4, IL-5, IFN-γ, IL-12, IL-10, and chemokines MCP-1, MIP-1β, and RANTES were measured using a multiplex Luminex assay. The data are expressed as the mean concentration (pg/mL)±SEM observed in serum samples from 3 animals per group;

FIG. 5 shows that EBOV GP induces innate immune responses via TLR4. C57BL/6 mice were pre-treated with LPS-RS (5 μg per mouse) for 2 days prior to co-administration of 100 μg GP and 10 μg LPS-RS per mouse. Serum levels of multiple cytokines and chemokines were measured using a multiplex Luminex assay. The data are expressed as the mean concentration (pg/mL)±SEM from at least 3 animals per group. Significance of differences between sera from GP and GP+LPS-treated mice was analyzed by two-way ANOVA followed by a Sidak's multiple comparison test. *p<0.05, **p<0.01;

FIGS. 6A-6B shows that GP-associated innate immune response affects homing of immune cells into the draining lymph nodes. C57BL/6 mice were administered intraperitoneally with PBS (C), GP alone (100 μg per mouse), or GP+LPS-RS. Inguinal lymph nodes (LNs) were harvested at 24 h after treatment, and disrupted single cell suspensions were stained using fluorochrome-conjugated antibodies specific for CD4⁺, CD8⁺, CD11b⁺, or CD11c⁺ and evaluated by flow cytometry. (A) Live cells were determined according to the size and granularity on the FSC vs. SSC histogram, and different subsets of cells were analyzed gated on live cells. CD11b⁺ or CD11c⁺ cells were measured in the cell population that was negative for CD4 and CD8. The figure is representative of three independent experiments. (B) The percentages of CD4⁺, CD8⁺, CD11b⁺, and CD11c⁺ cells in the LNs of control, GP or GP+LPS-RS-treated mice are expressed as mean ±SEM of three independent experiments in flow cytometry (n=3 per group). Significance of differences between treatments was analyzed by one-way ANOVA followed by a Tukey's multiple comparison test. **p<0.01, ***p<0.001, ****p<0.0001;

FIGS. 7A and 7B show that EBOV GP induces the phenotypic maturation of mouse BMDMs. BMDMs from C57BL/6 mice were exposed to 10 μg/mL EBOV GP or 100 ng/mL LPS for 2 days. The cells were washed and stained with fluorochrome-conjugated anti-CD40 and CD80 antibodies, and the surface expression of costimulatory molecules was assessed by flow cytometry. (A) One representative is presented in single parameter histograms (gray dotted line: control; black solid line: GP or LPS; gray shade: FMO). MFI (mean fluorescence intensity) values are shown as mean±SEM of two independent experiments in flow cytometry profiles. Positive cells exhibit a MFI greater than the value of FMO. (B) The percentages of CD40⁺ and CD80⁺ cells are expressed as mean±SEM of duplicate measurements in bar graphs;

FIG. 8 shows the cytokines and chemokines induced by EBOV GP in outbred Swiss Webster mice. Swiss Webster mice were administered with EBOV GP (100μg/mouse) or equal amount of total protein prepared from the cell culture supernatant of Drosophila S2 cells (NULL control) via the i.p. route. Mouse serum was collected at 6 and 24 hours after administration and levels of cytokines TNF-α, IL-1(3, IL-6, IL-2, IL-4, IL-5, IFN-y, IL-12, IL10, and chemokines MCP-1, MIP-1β, and RANTES were measured using a multiplex Luminex assay. The data are expressed as the mean concentration (pg/mL)±SEM in serum samples from 3 to 6 animals per group;

FIG. 9 shows the production of type I IFN in mouse serum after treatment with EBOV GP. C57BL/6 mice were administered with GP (100 μg per mouse), GP+LPS-RS or total protein prepared from Drosophila S2 cell culture supernatant (NULL). Mouse serum was collected at 24 hours, and the levels of IFN-I3 were measured using a commercially available mouse IFN-13 serum ELISA kit. The data is expressed as the mean concentration (pg/mL)±SEM in serum samples from 2 to 6 animals;

FIGS. 10A-10F show the expression of costimulatory molecules on mouse DCs after treatment with EBOV GP and GP subunits. BMDCs prepared from C57BL/6 mice were exposed to medium (C), EBOV GP, GP1, and GP2 as well as S2 (NULL control), total protein prepared from the cell culture supernatant of Drosophila S2 cell, or LPS for 30 h. The expression of CD40, CD80, and CD86 on the cell surface were evaluated by flow cytometry and expressed as (A-C) percentages of positive cells and (D-F) median fluorescence intensity (MFI). Significance of differences between treatments was analyzed by one-way ANOVA **p<0.01, ***p<0.001, ****p<0.0001;

FIGS. 11A-11B show EBOV GP1 induces higher gene expression of pro-inflammatory cytokines in mouse macrophages. BMDMs prepared from C57BL/6 mice were treated with EBOV GP, GP1, GP2, S2 (NULL control), or LPS. Total cellular RNA was extracted. The change in the mRNA levels of (A) TNF-α and (B) IL-1β at 2 and 6 hours after treatment was analyzed by real-time quantitative reverse transcription PCR (qRT-PCR) using specific primers. The data were normalized to GAPDH mRNA and fold-changes were calculated. The results are presented as mean±SEM of at least two independent treatments analyzed in duplicate wells;

FIG. 12 shows the glycosylation status of recombinant EBOV GP. SDS PAGE gel (10%) run under reducing conditions demonstrates that enzymatic deglycosylation of 1 μg purified, recombinant GP (purified protein: lane 1) with PNGase F results in reduction in size of both GP1 (top band) and GP2 (bottom band) (lane 2: 1 μg GP, PNGase treated). The GP2 band becomes more defined due to expected reduction in structurally distinct subspecies. Lane 3: Further deglycosylation using the EDEGLY kit (Sigma, St. Louis, Mo.) which used debranching enzymes Sialidase A, β(1-4)-Galactosidase and β-N-Acetylglucosaminidase in addition to PNGase F did not result in further size reduction of the two GP-subunits. This suggests that no O-linked glycosylation of the polypeptide has occurred, the expected finding for expression in Drosophila S2 cells;

FIG. 13 depicts a Coomassie stained SDS-PAGE gel (4-12%) showing Molecular weight standard MW (sizes in kDa), followed by 1 μg each of single step IAC purified EBOV GP (two batches, E1 & E2), MARV GP (M) and SUDV GP (S);

FIG. 14 depicts identical Western-blot panels of purified E-GP, M-GP and S-GP were generated and probed by EBOV, MARV and SUDV-specific monoclonals demonstrating that these recombinant antigens are virus specific;

FIG. 15 is a chromatogram showing size-exclusion-chromatography of IAC-purified E-GP. The graph with two narrow peaks shows A280 extinction; retention times of the two peaks represent trimers (right peak) and dimers of trimers (left peak), respectively. This finding was also verified by EGS-crosslinking prior to SDS-PAGE (data not shown);

FIG. 16A depicts the overall structure of EBOV GP. (a) Molecular surface of the GP trimer viewed on its side and down its threefold axis. Monomer A has an intensity profile according to its subdomains: GP1 base; GP1 head; GP1 glycan cap; GP2 N terminus; GP2 internal fusion loop; and GP2 HR1 (Lee J E, Saphire E O: Neutralizing ebolavirus: structural insights into the envelope glycoprotein and antibodies targeted against it. Curr Opin Struct Biol 2009, 19(4):408-417);

FIG. 16B is a schematic depicting the identification of various structural subunits of EBOV GP and amino acid positions;

FIGS. 17A-17C depict results of experiments testing induction of innate immune response and expression of costimulatory molecules. (A) C57BL/6 mice (n=3) were pre-treated with MARV GP (100 μg per mouse) via i.p route. Levels of multiple cytokines were measured in the sera using a multiplex Luminex assay and expressed as the mean concentration (pg/mL)±SEM. (B) Both Balb/c and (C) C57BL/6 mice were used to determine the surface expression of costimulatory molecules by flow cytometry. The cells were stained with fluorochrome conjugated anti-CD40 and CD80 antibodies, and the MFI (mean fluorescence intensity) values and percentages of CD40+ and CD80+ cells were assessed using gating strategy as described previously (Lai C Y, Strange D P, Wong T A S, Lehrer A T, Verma S: Ebola Virus Glycoprotein Induces an Innate Immune Response In vivo via TLR4. Front Microbiol 2017, 8:1571). The data is expressed as mean±SEM of at least two experiments. Error bars indicate SEM. *,p<0.05, **p<0.01, ***p<0.001, ****p<0.0001;

FIGS. 18A-18B depict results of experiments testing induction of cytokine responses. (A) BMDMs from Balb/c were pre-treated with 1 μg of EBOV GP, GP1 or supernatant from S2 cells and expression of cytokines at 24 hrs after treatment was determined using qRT-PCR. (B) Human monocytes derived THP-1 cells were treated with 1 μg/mL purified EBOV GP and at different time points after treatment and mRNA expression of TREM-1 was determined using qRT-PCR. Data expressed as fold-increase +SEM as compared to untreated controls after normalizing to GAPDH levels;

FIG. 19 shows a method for characterization of the cell- and domain-specific innate immune response to filovirus GP;

FIG. 20 depicts survival in comparison to untreated control animals, and shows that while pre-treatment did not improve survival in this uniformly lethal challenge experiment, co-treatment protected 2/10 BALB/c and 3/10 C57BL/6. The survival curves were significantly different from control groups (p<0.05 using the Gehan-Breslow-Wilcoxon test); and

FIGS. 21A-21B show that EBOV GP significantly enhanced the frequency of GC and Tfh cells in the spleen. Splenocytes from each group of mice were stained for markers of GC and Tfh cells and analyzed by flow cytometry. Doublets were excluded by FSC-A/FSC-H gating strategy and dead cells (cells that take up Invitrogen Live/Dead fixable yellow dye) were excluded from our analysis. (A) GC B cell frequencies from individual mice are shown after one dose (Day 16) of immunization. (B) Tfh cells were defined as CXCR5+PD-1+ T cells within CD3+CD4+ T cell gate. Tfh cell frequencies from individual mice are shown after first (Day 16) and second dose (Day 28) of immunization.

### DETAILED DESCRIPTION OF THE INVENTION

The disclosures of any publications, patents, and patent applications referred to herein are hereby incorporated by reference in their entireties into this application to the same extent as if each individual publication, patent, or patent application was specifically and individually indicated to be incorporated by reference. The instant disclosure will govern in the instance that there is any inconsistency between the publications, patents, or patent applications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

The present invention is based in part on the discovery that antigens such as glycosylated polypeptides that correspond to one or more subunits of glycoprotein (e.g., of EBOV) induce an innate immune response, even when administered in the absence of an adjuvant.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

As used in this specification and the appended claims, the singular forms “a”, “an”, and “the” include plural references unless the context clearly dictates otherwise. Thus, for example, references to “the method” includes one or more methods or steps of the type described herein, which will become apparent to persons skilled in the art upon reading this disclosure.

The term “about” or “approximately” are defined as being close to as understood by one of ordinary skill in the art, and in one non-limiting embodiment the terms are defined to be within 10%, preferably within 5%, more preferably within 1%, and most preferably within 0.5% of the qualified value.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5% of the qualified value.

The term “effective” as that term is used in the specification and/or claims, means adequate to accomplish a desired, expected, or intended result.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example from Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes such as Zn-protein complexes; non-ionic surfactants such as TWEEN™, PLURONICS™, or polyethylene glycol (PEG); or combinations thereof.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002), the entire contents of which are herein incorporated by reference.

The terms “administration of” and “administering a” compound should be understood to mean providing a compound of the disclosure or pharmaceutical composition to a subject. An exemplary administration route is intravenous administration. In general, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, sub capsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The compositions of the present invention may be processed in a number of ways depending on the anticipated application and appropriate delivery or administration of the pharmaceutical composition. For example, the compositions may be formulated for injection.

The compounds can be administered in various modes, e.g. orally, topically, or by injection. In some embodiments, the compounds are administrated by injection. The precise amount of compound administered to a patient can be determined by a person of skill in the art. The specific dose level for any particular patient will depend upon a variety of factors including the activity of the specific compound employed, the age, body weight, general health, sex, diets, time of administration, and route of administration.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Although no FDA approved vaccine or treatment against Ebola virus disease (EVD) is currently available, Ebola virus glycoprotein (GP) is a major antigen for candidate Ebola vaccines. However, immune responses induced by EBOV GP in the absence of viral vectors or adjuvants have not been fully characterized in vivo. Our studies demonstrated that immunization with highly purified recombinant GP in the absence of adjuvants induced a robust IgG response and partial protection against EBOV infection suggesting that GP alone can induce protective immunity. We investigated the early immune response to purified EBOV GP alone in vitro and in vivo. We show that GP was efficiently internalized by antigen presenting cells and subsequently induced production of key inflammatory cytokines. In vivo, immunization of mice with EBOV GP triggered the production of key Th1 and Th2 innate immune cytokines and chemokines, which directly governed the recruitment of CD11b⁺ macrophages and CD11c⁺ dendritic cells to the draining lymph nodes (DLNs). Pre-treatment of mice with a TLR4 antagonist inhibited GP-induced cytokine production and recruitment of immune cells to the DLN. EBOV GP also upregulated the expression of costimulatory molecules in bone marrow derived macrophages suggesting its ability to enhance APC stimulatory capacity, which is critical for the induction of effective antigen-specific adaptive immunity. Collectively, these results provide the first in vivo evidence that early innate immune responses to EBOV GP are mediated via the TLR4 pathway and are able to modulate the innate-adaptive interface. These mechanistic insights into the adjuvant-like property of EBOV GP may help to develop a better understanding of how optimal prophylactic efficacy of EBOV vaccines can be achieved as well as further explore the potential post-exposure use of vaccines to prevent filoviral disease.

EBOV initially targets mononuclear phagocytic cells (monocytes and macrophages) and dendritic cells (DCs) that play a critical role in virus dissemination and spread to the liver, spleen and other tissues and cell types. Studies of non-human primates (NHPs) and in vitro models showed that EBOV infection triggers monocytes and macrophages to induce strong innate immune responses including production of several inflammatory cytokines and chemokines such as interleukin (IL)-1β and IL-6, and tumor necrosis factor (TNF), but fails to activate DCs. In vitro studies using virus-like particles (VLPs) have demonstrated that macrophages and DCs can be activated by GP and produce cytokine and chemokines through the TLR4 signaling pathway, which further supports T cell proliferation. A report showed that shed GP activates macrophages and DCs, which may cause a massive release of pro- and anti-inflammatory cytokines and affect vascular permeability. In addition, EBOV or GP can enhance monocyte maturation, which promote virus infection, further causing the death of T lymphocytes. However, GP-induced innate immune responses have not been fully characterized in vivo.

### Antigens

In one aspect, the present disclosure provides a composition that includes a portion (e.g., a glycosylated polypeptide) of EBOV glycoprotein (GP) or the full-length GP.

EBOV GP, in an embodiment, includes amino acids 33-647 of Zaire ebolavirus, Mayinga strain, GP glycoprotein. Genomic sequence of this strain can be found at Genbank accession number NC_002549.

In some embodiments, the glycosylated polypeptide has an amino acid sequence that is at least 95% identical to an amino acid sequence range among 1-501,33-501,33-201, 201-309,309-501, and 502-676 of SEQ ID NO: 2. The polypeptides may be combined to create multivalent antigens. In some embodiments, various GP proteins of different filoviruses may be combined to create a multivalent formulation. In an embodiment, the used antigen is GP1 (e.g., residues 1-501 of SEQ ID NO: 2). In an embodiment, the used antigen is a truncated GP1 (e.g., residues 33-501 of SEQ ID NO: 2, which correspond to the GP1 that is the native form due to the secretion signal being removed. We may use a longer version as well. In some embodiments, the GP protein is encoded by a DNA sequence provided in SEQ ID NO: 1, and may undergo additional processing before attaining the antigenic protein form that is used herein.

In some embodiments, the glycosylated polypeptide has at least 100 amino acid residues of GP. In some embodiments, the glycosylated polypeptide has at least 50 amino acid residues of GP. In some embodiments, the glycosylated polypeptide has at least 20,30, 40 amino acid residues of GP.

The used antigens, in many embodiments, retain a conformation that mirrors that of the native GP subunit(s). The glycosylation pattern of the antigens in some embodiments corresponds to the N-linked glycosylation pattern of the native GP subunit(s).

Using the GP, or a portion of it, improves the safety of the antigen, at least because the antigen is non-replicative. When a portion of the GP is used, in some embodiments, that portion corresponds to a structural subunit of the GP.

### Adjuvants

In one aspect, the present disclosure provides a composition that is administered in the absence of an adjuvant. Therefore, no adjuvant is needed.

However, adjuvants need not be excluded from the compositions. Therefore, the compositions, in some embodiments, may also be used in the presence of adjuvants. Some adjuvants that may be used include CoVaccine HT™ (Protherics Medicines Development Ltd.; London, UK), ISA51 (Seppic; Fairfield, N.J.), Ribi R-700 (Sigma-Aldrich; St. Louis, Mo.), GPI-0100, GLA-SE, and DepoVax.

### Production of the Antigens

Also provided as aspects of the present invention are methods of producing the antigens.

The expression of GP subdomains with Drosophila S2 cell approach is novel. The Drosophila S2 expression system has been successfully utilized to develop several flavivirus vaccines including dengue virus and West Nile virus. Compared to other expression platforms such as E. coli or various mammalian cell culture systems, immunoaffinity-purified Drosophila S2 expressed antigens have been demonstrated to properly fold and maintain native-like characteristics with suitable adjuvants. Relatively high yields support future commercial production. Development of recombinant EBOV subunit vaccines using the Drosophila system enables production of sufficient quantities of highly purified GP with native conformation. Additionally, we utilize monoclonal antibodies to produce highly purified individual GP subdomains. This is the first systematic study to produce individual structural domains from EBOV GP with a focus on comparing the innate and adaptive immune responses elicited by these GP subdomains.

Therefore, the antigens can be expressed in a Drosophila S2 system, which provides N-linked glycosylation, and then purified via IAC using monoclonal antibodies. Using this approach, full or partial GP subunit antigens can be produced. After the production, the antigens may be stored either in lyophilized form or in liquid form, as demonstrated by the Examples 6, 7, and 8.

### Uses of the Antigens

Also provided as aspects of the present invention are methods of using the antigens.

In-depth analysis of innate and adaptive immune responses to recombinant GP is novel. The study uses model systems including relevant mouse immune cells including monocytes, dendritic cells (DC) and NK cells, human immune cells and both BALB/c and C57BL/6 strains of mice to comprehensively address proposed study questions. The approach of using single cell RNA-Sequencing to characterize different PRR pathways activated by GP and their role in shaping the adaptive immunity is novel. Further, this is the first study to illustrate the GP-specific responses on protective immunity with a particular focus on consistent immunogenicity and durability in subjects.

The antigens can be used before an infection, for example to protect against future infection. This is similar to a conventional vaccination strategy. Initially stimulated innate immune response provides quick protection, while a subsequent adaptive immune response further protects against the ongoing or subsequent infections. The antigens/compositions can also be used post-infection, to provide additional immunity against an infection. Furthermore, the compositions can also be used to protect against non-infectious conditions, such as cancer. Because the compositions boost an innate immune response (and not only an adaptive one), they are beneficial against non-infectious conditions as well. This makes their use broader than what the source of the antigen(s) may indicate. As such, their use is not limited to filovirus infections.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

The following describes studies demonstrating that Ebola virus glycoprotein induces an innate immune response in vivo via the TLR4 pathway.

The results from a recent human EBOV vaccine clinical trial in Guinea are encouraging and indicate that rVSV-ZEBOV is safe and highly efficacious in preventing EVD when delivered via a ring vaccination strategy during an outbreak. The results from the phase 3 vaccination trial using rVSV-ZEBOV showed that this vaccine induced protection as quickly as 6 days after administration even before robust IgG responses were generated. We thought that an adaptive response may not be the only way by which EBOV GP can confer protective immunity. Based on prior outbreak reports and recent clinical studies, it appears to us that the difference between EVD survivors and fatalities lies in the early immune responses elicited during the virus infection that may also explain protection in the recently used ring vaccination approach.

Both innate and adaptive immune responses are important for robust vaccine-induced protection. Vaccine adjuvants typically function to boost the innate immune response; therefore, understanding of the specific innate immune response induced by the antigen alone is important to optimize antigen and adjuvant formulations and dosing schedules. We investigated whether purified GP alone in the absence of other viral components and adjuvants can activate APCs and induce innate responses including the production of inflammatory cytokines using a murine model. We show here that exposure of mouse bone marrow-derived macrophages (BMDMs) to EBOV GP induced robust production of cytokines TNF-α and IL-1β but not of type I IFN. In vitro experiments using BMDMs demonstrated that GP was efficiently internalized by APCs and up-regulated expression of co-stimulatory molecules, suggesting that GP can enhance the stimulatory capacity of APCs, which is critical for induction of effective antigen-specific adaptive immunity. In vivo, GP also triggered production of multiple cytokines and chemokines and we further demonstrated that the GP-induced cytokine response occurs via activation of TLR4 signaling, directly affecting the recruitment of immune cells to the draining lymph nodes. Collectively, our data provide the first in vivo evidence that GP-induced innate immunity is via TLR4 and modulates key immune events critical for early control of the virus as well as fine tuning the innate-adaptive interface.

### Materials and Methods

### Ethics Statement

This study was carried out in accordance with the recommendations in the Guide for the Care and Use of Laboratory Animals of the National Institutes of Health. The protocol was approved by the Institutional Animal Care and Use Committee at the University of Hawaii, and efforts were made to minimize animal suffering.

### Recombinant Protein

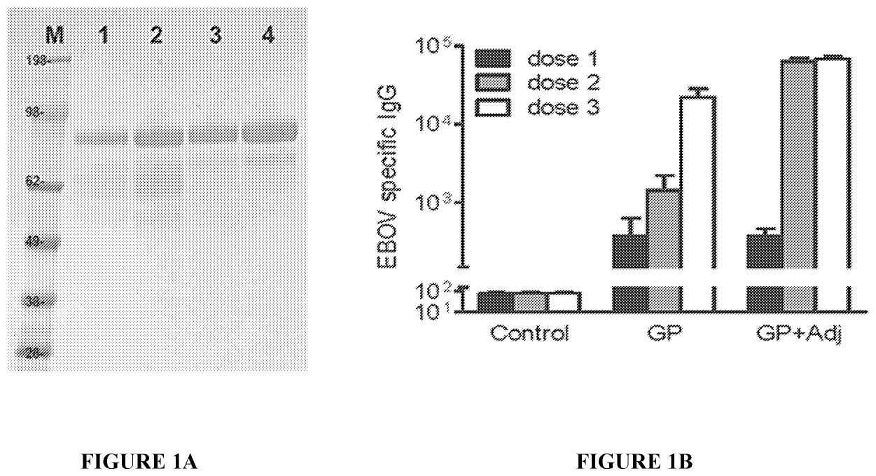

Recombinant EBOV GP was produced from insect cells and purified using immunoaffinity chromatography. To further increase the level of purity, protein used for our studies was subjected to an additional purification step via size-exclusion chromatography using a HiLoad 16/600 Superdex 200 prep grade column (GE Healthcare Life Sciences, Piscataway, N.J.) equilibrated in phosphate buffered saline, pH7.4 (FIG. 1, panel A). FITC labeling of purified EBOV GP was performed using the Pierce FITC Antibody Labeling Kit (Thermo Fisher Scientific) according to manufacturer's instructions. Briefly, protein prepared in borate buffer (50 mM sodium borate, pH 8.5) was added to FITC reagent and mixed by pipetting up and down. After incubation for 60 min at room temperature, the labeling reaction was added to the spin column containing a purification resin to remove unbound FITC. After thorough mixing, the purified FITC-labeled protein was eluted by centrifugation of the spin columns for 30-45 s at 1,000 g.

### Cell Culture

Human THP-1 cells, a monocytic cell line, obtained from the American Type Culture Collection (ATCC) were grown in RPMI culture media (Sigma) containing 10% fetal bovine serum (FBS) (GE Healthcare Life Sciences), 1% penicillin/streptomycin (Pen/Strep) (Invitrogen), and 0.0006% β-mercaptoethanol (Bio-Rad). Mouse bone marrow-derived macrophages (BMDMs) were obtained by differentiation of bone marrow progenitor cells. Briefly, bone marrow cells were isolated from the pelvis, femurs, and tibias of wild type C57BL/6 or BALB/c mice. Red blood cells (RBC) were lysed using BD Pharm Lyse™ lysing solution (BD Biosciences), and the cells were cultured for 7 days in DMEM (Sigma) supplemented with 10% FBS, 1% Pen/Strep, and 14% (v/v) L929-conditioned medium which contains macrophage colony-stimulating factor (M-CSF) secreted by L929 cells. Culture medium was replaced on day 3 and 6 of culture, and fully differentiated mouse macrophages were used for experiments on day 7 of culture.

Analysis of Cytokine Gene Expression In vitro by Quantitative Real-Time PCR

THP-1 cells and mouse BMDMs were treated with EBOV GP (1 μg/ml), and the cell culture supernatants and cell lysates were harvested at 2, 6, and 24 h after treatment for further analysis. The total cellular mRNA was extracted and reverse transcribed to cDNA using the NucleoSpin® RNA kit (Macherey-Nagel) and iScript cDNA synthesis kit (Bio-Rad). The synthesized cDNA was subjected to qPCR using iQ SYBR Green Supermix (Bio-Rad) and cytokine gene-specific primers. The fold change of mRNA levels in GP treated cells was calculated compared to mock after normalizing to the GAPDH gene.

In vitro Assay for Antigen Uptake By Mouse BMDMs

BMDMs prepared from C57BL/6 mice were re-plated in a non-tissue culture treated 24-well plate at a density of 5×105 cells per well on day 6 of differentiation. Twenty-four hours later, the cells were treated with medium only (control), or 10 μg/ml of GP-FITC. After 30 min of incubation at 37° C., the reaction was stopped by washing with ice cold 1× PBS. The adherent cells were detached by incubation with Cellstripper solution (Corning) at 37° C. for 30 min, washed, and fixed with 4% paraformaldehyde in PBS at 4° C. for 30 min. The purity of differentiated BMDMs was determined by staining of the cells with APC-conjugated anti-mouse CD1lb antibody (clone M1/70) (eBioscience). GP-FITC+ or CD11b+ cells were evaluated on a FACSCalibur flow cytometer (BD Biosciences). Flow data were analyzed using FlowJo software (TreeStar Inc).

### In Vitro Assay For the Expression of Costimulatory Molecules in Mouse BMDMs

Differentiated BMDMs prepared from C57BL/6 mice were re-plated in a non-tissue culture treated 24-well plate at 5.0×105 cells per well. The cells were treated with medium only (control), 10 μg/ml purified recombinant EBOV GP, or 100 ng/ml of LPS (Invivogen). On day 2 of treatment, the cells were detached using Cellstripper solution (Corning), washed with cold PBS supplemented with 2% FBS and Fc-blocked by incubation with anti-mouse CD16/CD32 antibody (eBioscience). The cell surface markers CD40 and CD80 were stained using the following antibodies: APC-conjugated anti-mouse CD40 (clone 1C10) and PE-conjugated anti-mouse CD80 (clone 16-10A1) (eBioscience). The purity of differentiated macrophages was confirmed by staining of the cells with FITC-conjugated anti-mouse CD1 lb antibody (clone M1/70) (eBioscience). After incubation with antibodies, cells were washed, fixed with 4% paraformaldehyde in PBS, and analyzed using a FACSCalibur flow cytometer (BD Bioscience). Fluorescence minus one (FMO) samples were prepared for each fluorochrome to facilitate gating. The data were analyzed using Flowjo software (Treestar Inc.).

### Mouse Experiments

Wild-type C57BL/6, and BALB/c, and Swiss Webster mice were bred in our laboratory using breeding stock obtained from Jackson Laboratories and Taconic Farms, Inc., respectively. This study was specifically approved by the University of Hawaii Institutional Animal Care and Use Committee (IACUC), and conducted in strict accordance with guidelines established by the National Institutes of Health and the University of Hawaii IACUC. Seven to eight-week old mice were administered EBOV GP (100 μg per mouse) intraperitoneally (i.p.). In some experiments, mice were pre-treated with a TLR4 antagonist, ultrapure lipopolysaccharide from the bacterium Rhodobacter sphaeroides (LPS-RS) (InvivoGen) at the dose of 5 and 10 μg per mouse via i.p. route on day −2 and −1 before administration of GP, respectively. A group of mice was also administered an equal amount of total protein (100 μg per mouse) of the cell culture supernatant from Drosophila S2 cells prepared by the same procedure as the GP (NULL control). Sera were collected at 6 and 24 h after GP administration for cytokine analysis. For lymph node cell subset analysis, mice were euthanized at 24 h, and inguinal lymph nodes were collected.

### Flow Cytometric Analysis of Cell Subsets in the Draining Lymph Nodes

Inguinal lymph nodes (LNs) obtained from C57BL/6 mice treated with PBS (control), EBOV GP or GP+LPS-RS were placed in 1× PBS and single cell suspensions were generated by mechanical disruption with a syringe plunger and passing through a 70 μm cell strainer. The total number of live cells was calculated by trypan blue exclusion using a hemocytometer. The cells were incubated with anti-mouse CD16/CD32 antibody in staining buffer (lx PBS supplemented with 2% FBS) to minimize non-specific binding, and stained with the following: PE-Texas red-conjugated anti-mouse CD4 (clone GK1.5), APC-conjugated anti-mouse CD8a (clone53-6.7), PerCPCy5.5-conjugated anti-mouse CD11b (clone M1/70), and PE-conjugated anti-mouse CD11c (clone N418). Antibodies were purchased from eBioscience. Different subsets of cells were analyzed on a FACSAria flow cytometer (BD Biosciences) and the data were analyzed using Flowjo software (TreeStar).

### Measurement of Cytokines and Chemokines in Mice

The levels of multiple cytokines and chemokines in the sera from mice administered with EBOV GP in the presence or absence of LPS-RS were measured using a Bio-Plex Pro™ mouse cytokine standard 23-plex, group I kit (Bio-Rad). Mouse serum samples at 1:3 dilution were assessed for the production of the following cytokines: IL-1α, IL-1β, IL-2, IL-3, IL-4, IL-5, IL-6, IL-9, IL-10, IL-12p40, IL-12p70, IL-13, IL-17A, Eotaxin, G-CSF, GM-CSF, IFNγ, MCP-1, MIP-1α, MIP-1β, RANTES, and TNFα according to manufacturer's instructions. Plates were read using the Luminex 200 xMAP system (Millipore) and data were analyzed using the Luminex xPONENT software (Millipore). The level of beta interferon (IFN-β) in mouse sera was measured using the VeriKine mouse interferon beta ELISA kit (PBL Assay Science) according to manufacturer's instructions.

### Statistical Analysis

Significant differences in the serum levels of cytokines and chemokines or LN cell subsets between groups of mice were determined by ANOVA tests using GraphPad Prism version 7.0 (GraphPad software, San Diego, Calif.). P values of <0.05 were considered significant.

### Results

Immunization with EBOV GP Alone Induces Strong Antibody Production and Affords Protection against Lethal Viral Challenge in Mice

Mouse models have proven to be useful tools to understand immune responses to filovirus infection and evaluate vaccines and antiviral compounds. Our previous study used a mouse model to evaluate the potential of recombinant EBOV proteins expressed in stably transformed Drosophila S2 cell lines to protect against EBOV infection. We demonstrated that while 3 doses of purified recombinant EBOV GP along with adjuvant completely protected mice challenged with 100 PFU of mouse adapted EBOV, mice immunized with GP alone showed partial protection, with survival of 7 of 10 mice following lethal challenge. To further understand the association between protection and antibody response to EBOV GP alone and GP+adjuvant, we evaluated the kinetics of GP-specific IgG antibodies in BALB/c mice immunized with 10 μg of GP or GP+adjuvant via the subcutaneous route, followed by two booster doses at 4 week intervals post primary immunizations. As seen in FIG. 1, all animals immunized with GP+adjuvant demonstrated detectable EBOV-specific antibodies at week 3 after primary immunization that increased sharply after the second dose and remained high after the third dose. In comparison, animals immunized with GP alone developed antibody titers after the first dose which were similar to the GP+adjuvant group, but increased more gradually after the second and third doses. The difference between the GP-specific endpoint IgG titers after the third dose in both groups was not statistically significant. These results suggested that GP alone can elicit potent IgG titers and a protective immune response against EBOV infection.

EBOV GP Is Efficiently Endocytosed by Mouse Macrophages and Induces Upregulation of Cytokine mRNA Transcription In Vitro

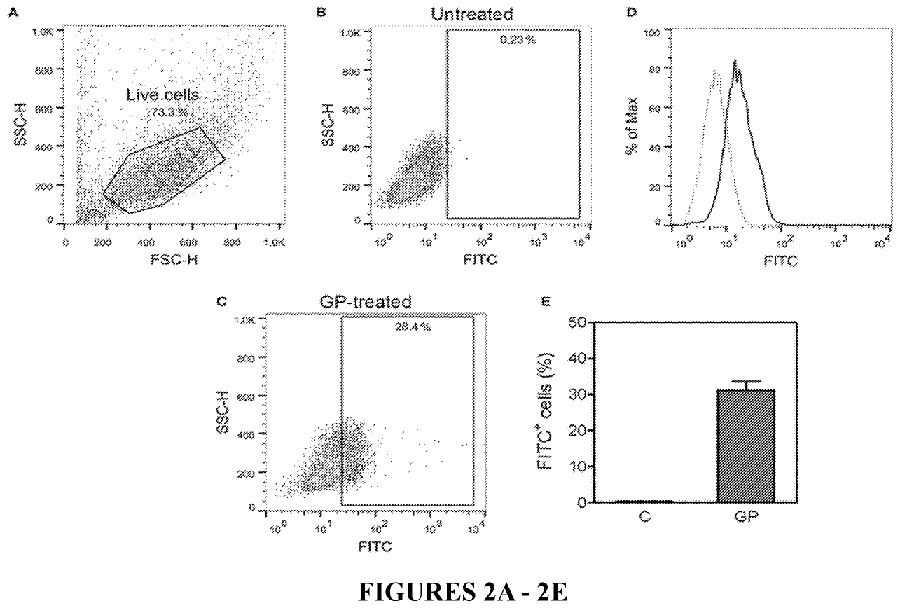

Since monocytes and macrophages are the initial cell targets of EBOV and the main immune cells that secrete cytokines during EBOV infection, we investigated whether GP can activate these cells and initiate immune responses. As antigen uptake is the first step toward activation of APCs, we determined the ability of BMDMs to internalize GP. The purity of differentiated BMDMs was determined as the percentage of CD11b+ cells and was observed to be >95% (data not shown). As shown in FIG. 2, GP was efficiently endocytosed by BMDMs and after 30 min of incubation, almost 28-36% of BMDMs were found to be GP-FITC positive.

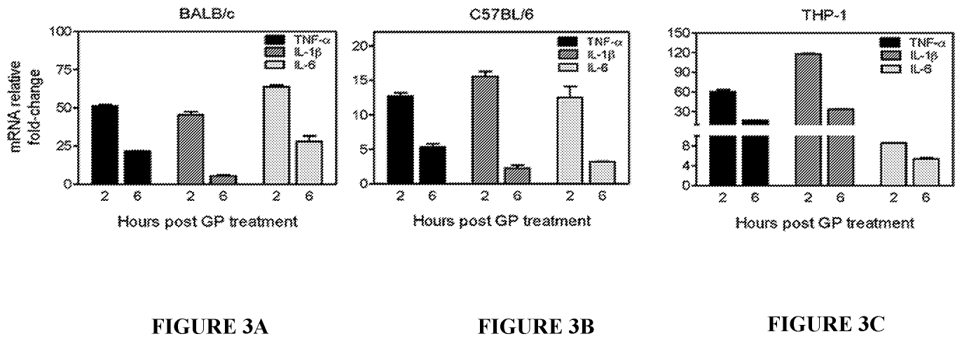

We further investigated whether internalization of GP also stimulates the induction of innate immune cytokines in mouse BMDMs. BMDMs derived from both C57BL/6 (Th-1 dominant) and BALB/c (Th-2 dominant) mice were exposed to EBOV GP, and the intracellular levels of mRNA transcripts coding for key inflammatory cytokines were analyzed at 2, 6, and 24 h post-exposure using quantitative real-time PCR. As shown in FIG. 3, panel A, pro-inflammatory cytokines TNF-α, IL-1β, and IL-6 were significantly induced (45-64-fold) at 2 h after GP exposure of BALB/c BMDMs, but decreased dramatically at 6 h, and reached basal levels at 24 h post exposure (data not shown). Similarly, GP triggered the transcription of TNF-α, IL-1β, and IL-6 genes in BMDMs of C57BL/6 mice within 2 h of exposure and the levels of the transcribed mRNAs also declined after 6 h of exposure (FIG. 3, panel B).

To ascertain whether EBOV GP also induces similar responses in human immune cells, we treated THP-1 cells, a human monocytic leukemia cell line extensively used to study human monocyte/macrophage functions, with 1 μg/mL of GP. As shown in FIG. 3, panel C, we observed a similar pattern of mRNA transcription of TNF-α, IL-1β, and IL-6 genes in THP-1 cells. TNF-α and IL-1β mRNAs increased by 60 and 120-fold, respectively within 2 h of exposure and declined after 6 h, reaching basal levels comparable to the untreated cells at 24 h after exposure (data not shown). Collectively, these results suggest that EBOV GP can stimulate induction of important innate immune cytokines, which are shown to be essential for the control of viral infection.

### EBOV GP Induces the Release of Innate Immune Cytokines and Chemokines In Vivo

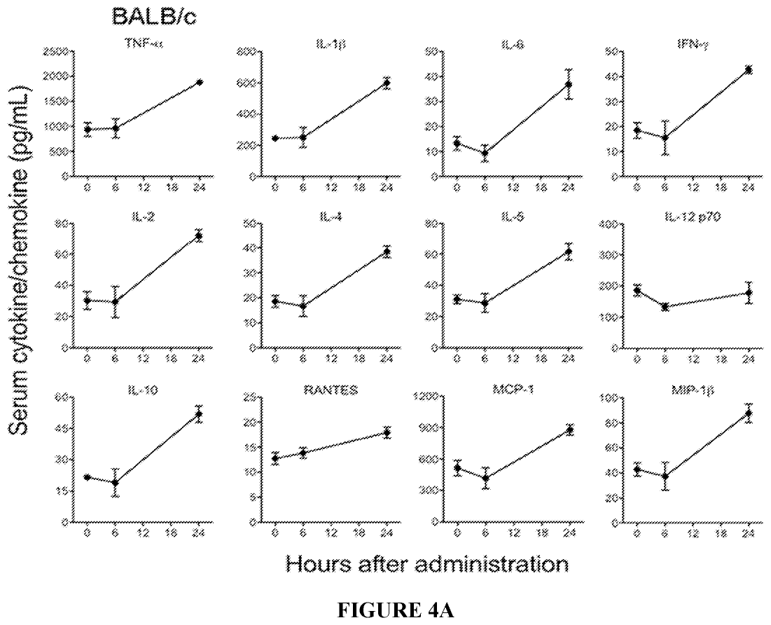

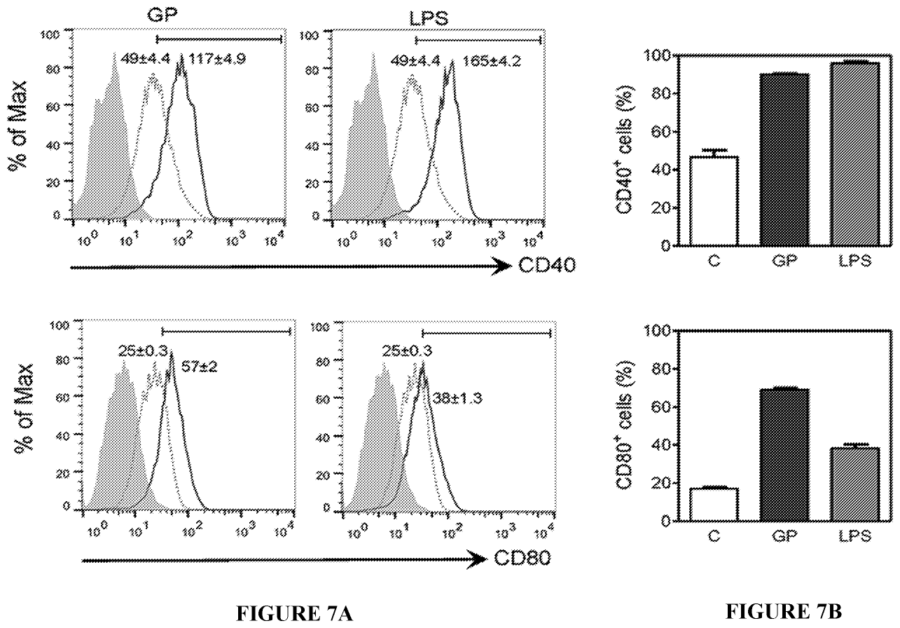

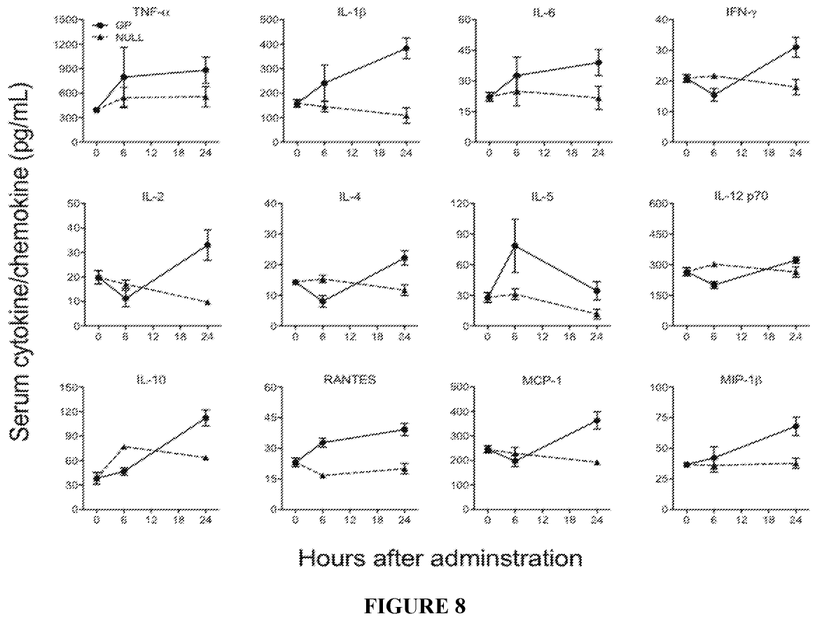

Cytokines and chemokines have been considered to be useful biomarkers for EVD in predicting disease outcome for survivors and non-survivors. Although other in vitro studies have looked into the innate immune response to EBOV VLPs and shed GP, the ability of GP to induce cytokines in vivo has not been determined so far. Therefore, we next characterized the in vivo immune response to GP after administration of 100 μg of EBOV GP to different strains of mice and subsequently analyzed the cytokine levels in serum at multiple time points. As shown in FIG. 4A, in GP-treated BALB/c mice we observed significant increases in the levels of key inflammatory cytokines (TNF-α, IL-1β, IL-6), chemokines (MCP-1, MIP-1β), T cell-derived cytokines (IL-2, IL-4, IL-5, IFN-γ), and of the anti-inflammatory cytokine IL-10. RANTES showed only a slight increase at 6 and 24 h while the level of IL-12 did not increase at all. On the other hand, levels of these GP-induced cytokines and chemokines (except for RANTES) were observed to be very high at both 6 and 24 h after EBOV GP treatment of C57BL/6 mice (FIG. 4B). We also compared the levels of multiple cytokines induced by GP and the equal amount of protein from the cell culture supernatant from Drosophila S2 cells as NULL control in Swiss Webster mice. Our results demonstrated that while GP induced multiple cytokines and chemokines, their levels in the mice administered with S2 supernatant were comparable with the basal levels observed in control mice injected with PBS thus suggesting that our results were specific to GP (FIG. 8). Since type I interferon (IFN) production is also an important feature of innate immunity to viruses, we further investigated if GP induces type I IFN in vivo. However, we did not observe any change in the levels of IFN-β in the sera of GP-treated mice as compared to controls (FIG. 9). Collectively, these results provide first in vivo evidence that GP is capable of inducing strong innate immune inflammatory responses in different strains of mice.

EBOV GP Triggers the Production of Inflammatory Cytokines through the TLR4 Pathway

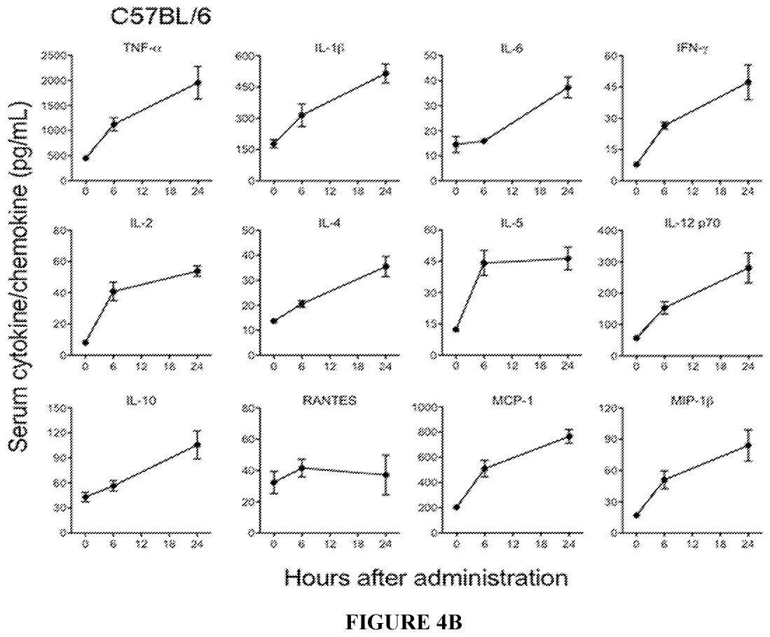

Since TLR4 is proposed to be one of the pathogen recognition receptors that binds to other secretory viral proteins including EBOV shed GP, we next tested whether TLR4 also mediates the innate immune responses to purified EBOV GP in vivo. The TLR4 signaling pathway was blocked using a commonly used TLR4 antagonist, LPS-RS, and the levels of GP-induced cytokines were compared to a non LPS-RS-treated group. As shown in FIG. 5, the levels of key cytokines and chemokines were attenuated in C57BL/6 mice pre-treated with LPS-RS. The differences for IL-6, IL-2, IL-5, IL-4, and chemokine MCP-1 between GP and GP+LPS-RS treated mice were statistically significant at 6 h, while TNF-α, IF-1β, IFN-γ, IL-12, and MIP-1β levels in GP+LPS-RS treated mice also showed a definite trend toward decreased levels as compared to GP treated mice. In contrast, RANTES production was increased by LPS-RS while the induction of the anti-inflammatory cytokine IL-10 was not affected by LPS-RS treatment. Taken together, our data suggest that TLR4 signaling is one of the major pathways involved in the inflammatory response induced by EBOV GP in vivo.

### EBOV GP Promotes the Homing of Immune Cells to the Draining Lymph Nodes in a TLR4-Dependent Manner

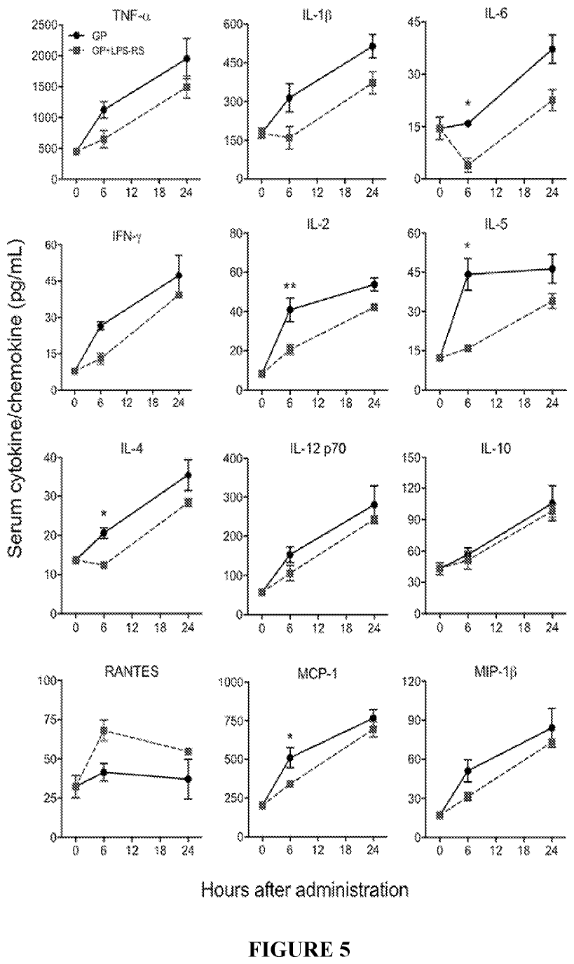

After vaccination or in the natural course of infection, production of cytokines and chemokines leads to the recruitment of activated APCs to the DLNs, where they activate the proliferation of antigen-specific T cells. Given that GP induced cytokines and chemokines in vivo, we next assessed whether GP also affects the homing of immune cells to DLNs. Single cell suspensions prepared from inguinal LNs of mice treated with EBOV GP were used to evaluate different cell subsets using flow cytometry. CD11b+ macrophages and CD11c+ DCs were analyzed after the exclusion of CD4+ and CD8+ cells. As shown in FIG. 6, in comparison to the mock group, significantly increased percentages of CD11b+ and CD11c+ cells were observed in the DLNs after GP treatment. However, pre-treatment of mice with LPS-RS reduced the recruitment of APCs to baseline. The percentage of CD4+ cells also increased in the DLNs of GP-treated mice, an effect that was also inhibited by LPS-RS. Collectively, our results suggest that following internalization of GP, activated APCs induced cytokine or chemokine production via TLR4 signaling, which subsequently enhanced the migration of immune cells to the DLNs and may promote the activation and proliferation of CD4 T cells in vivo.

### EBOV GP Enhances Macrophage Maturation

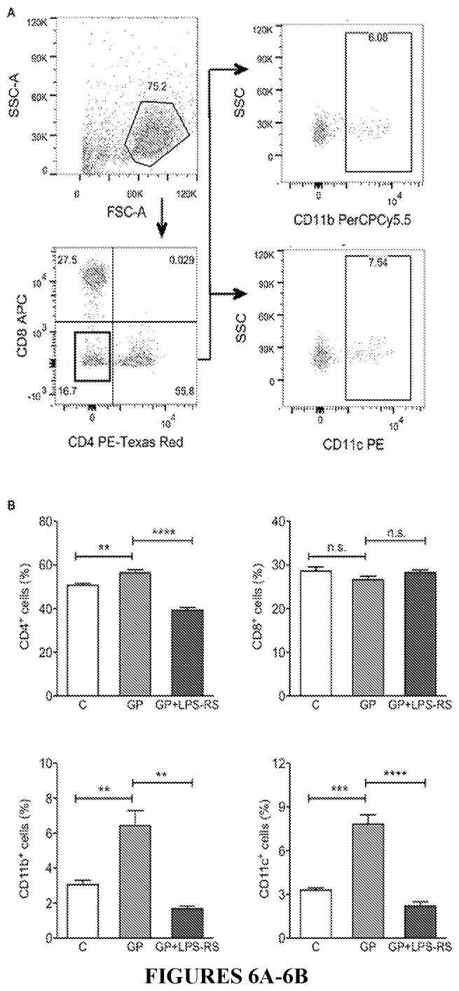

Upregulation of costimulatory molecules in APCs is necessary for antigen presentation and priming of T cell responses. Since our results showed that EBOV GP can trigger efficient uptake by macrophages and induce production of inflammatory cytokines, we next assessed whether GP can influence maturation of macrophages, an important event in fine-tuning the innate-adaptive interface. The effect of EBOV GP on the expression of costimulatory molecules CD40 and CD80 on mouse BMDMs was therefore determined by flow cytometry. As shown in FIG. 7, in GP-treated macrophages, surface expression of CD40 and CD80 was dramatically increased as compared to control cells, as indicated by the MFI values in flow cytometry profiles and the percentages of CD40+ and CD80+ cells. The expression levels of CD40 and CD80 in GP-treated BMDMs were comparable to those observed in BMDMs stimulated with LPS as positive control thus suggesting that GP can enhance the ability of APCs to induce T cell activation.

### Discussion

EBOV GP expressed in several vaccine approaches using viral vectors or virus-like particles has been shown to protect rodents and non-human primates from EBOV infection. Our previous study showed that highly purified recombinant EBOV GP in combination with matrix proteins VP24, and VP40 with or without adjuvants elicits both effective humoral and cellular immune responses, yielding up to 100% protection in mouse models. Purified proteins alone are generally only weakly immunogenic and need to be combined with adjuvants to enhance T and B cell responses. Interestingly, our previous study also demonstrated that three immunizations with purified recombinant GP alone resulted in 70% protection in mice (Lehrer et al., Recombinant proteins of Zaire ebolavirus induce potent humoral and cellular immune responses and protect against live virus infection in mice, Vaccine 36(22): 3090-3100), suggesting that GP is capable of inducing protective immune responses against EBOV infection. Little is known about the mechanisms by which GP induces protection, however, available data from naturally infected and vaccinated individuals provide indirect evidence for the role of early immune responses in protection against EVD. It has been reported that EVD survivors, as compared to fatally infected patients, develop well-regulated, early, and stronger inflammatory responses, which are proposed to be crucial to control viral replication and induce specific adaptive immunity. Furthermore, rVSV-ZEBOV, currently the most advanced vaccine candidate, was shown to protect non-human primates even before appropriate adaptive immunity was induced (vaccination 3 and 7 days before EBOV challenge). However, the protection level was reduced when the vaccine candidate was given 20-30 min after viral challenge. A similar trend was observed in the human phase 3 ring vaccination trial in Guinea where it was shown that rVSV-ZEBOV offered substantial protection against EVD from as early as 6 days after vaccination.

Our current study investigated the early immune responses to EBOV GP and how these responses might affect antigen presentation and the innate-adaptive interface. We demonstrated that highly purified, recombinant EBOV GP was efficiently internalized by macrophages independent of adjuvants that led to the induction of several primary inflammatory cytokines including TNF-a, IL-6, and IL-1β in monocytes and macrophages. In vivo mouse studies confirmed that GP treatment increased serum levels of key cytokines and chemokines at early time points. Another important highlight of our data is that GP-induced inflammatory responses in vivo are mediated by the TLR4 signaling pathway, and seem to play an important role in the homing of immune cells to the draining lymph nodes. Finally, we showed that the treatment of macrophages with GP triggers expression of key markers of antigen presentation, suggesting that early inflammatory responses induced by GP may further promote the development of an effective adaptive response.

The early stages of EBOV infection, virus entry and fusion, are mediated by surface GP. The transcription of the EBOV GP gene leads to the synthesis of two mRNAs encoding different forms of GPs, the non-structural secreted GP (sGP), and the viral surface GP. The surface GP is composed of two subunits, GP1 and GP2, linked by a disulfide bond, and presents as a trimer on the EBOV surface. During EBOV infection, cleavage of surface GP by the cellular metalloprotease TACE results in the release of truncated surface GP (shed GP) from infected cells. Other studies have reported that large amounts of sGP and shed GP released from virus-infected cells are detected in the blood of infected patients and Guinea pigs, and have been associated with EBOV pathogenesis. The GP shed from virus-infected cells can bind and activate human dendritic cells and macrophages, leading to the release of pro- and anti-inflammatory cytokines and affecting vascular permeability. In comparison, sGP was not shown to activate macrophages, suggesting that various forms of GP induce unique immune responses which may lead to different disease outcomes. Based on size-exclusion chromatography and subsequent analysis of the oligomerization state using EGS-crosslinking, it is clear that the majority of the recombinant GP used in our study resembles the trimeric form of GP presented on viral particles or VLPs and is comparable to the GP produced by virally vectored Ebola vaccine candidates. Our previous study also shows that only N-linked glycosylation sites are processed in the GP1 and GP2 regions our GP protein, a typical observation for glycoproteins expressed in the Drosophila expression system.

Our data demonstrated that purified recombinant EBOV GP is capable of activating macrophages indicated by the expression of pro-inflammatory cytokines, TNF-α, IL-6, and IL-1β, which agrees with what was observed in other studies using VLPs. It has been shown that in vitro, human macrophages exposed to live Ebola virus or UV-inactivated virus produce high levels of pro-inflammatory cytokines and chemokines. In studies using non-human primates and guinea pigs, macrophages were also suggested to be one of the major cell targets of EBOV infection, and play a key role in dissemination of virus to other tissues. This causes extensive viral replication and tissue damage in multiple organs. The massive release of inflammatory cytokines and chemokines by EBOV infected macrophages has been associated with viral pathogenesis. In EBOV-infected patients, fatal infection was associated with high levels of IL-10 and IL-1RA, modest levels of TNF-α and IL-6, and non-detectable levels of IL-1β, MIP-1α, and MIP-1β, while survivors were characterized by high levels of TNF-α, IL-1β, and IL-6 in plasma. This striking difference in the key inflammatory cytokines between survivors and non-survivors suggests an association of early innate immune response with EVD outcome. We believe that our results demonstrate balanced cytokine and chemokine responses including high levels of TNF-α, IL-1β, IL-6, MIP-1α, and MCP-1 as well as anti-inflammatory cytokine IL-10 at early time points in GP-treated mice mimicking the pattern observed in EVD survivors.

Our data using LPS-RS provide the first in vivo evidence that GP induces the production of pro-inflammatory cytokines and chemokines via TLR4-mediated immune signaling. This agrees with previous in vitro studies that reported production of specific pro-inflammatory cytokines by Ebola VLPs containing GP in THP-1 cells and HEK293 cells stably expressing the TLR4/MD2 complex. Collectively, our in vivo and previous in vitro data support the hypothesis that TLR4 is one of the sensors for EBOV GP. However, recombinant EBOV GP in our in vivo experiments did not induce IFN-β secretion, suggesting that the induction of type I IFN and ISGs observed in the previous study might have been triggered by viral or cellular proteins other than GP co-purified during the VLP production.

Protein antigens as vaccine candidates are generally less immunogenic than particulate antigens due to size, degradation, non-specific targeting, poor uptake by APCs, and inability to activate APCs thus justifying the use of adjuvants to mediate these responses. In contrast, our findings show that recombinant EBOV GP alone without any adjuvant is able to directly trigger antigen uptake by macrophages and enhance the surface expression of costimulatory molecules CD40 and CD80, which may reduce the threshold necessary for subsequent T cell activation. Another interesting finding of our study is that the migration of activated APCs to the DLNs was TLR4-dependent. The collective response to GP including induction of inflammatory cytokines and APC activation appears to be similar to the mechanisms by which adjuvants enhance vaccine-induced protective immunity. For instance, AS04, a licensed adjuvant used in the human papilloma virus (HPV) vaccine Cervarix™, has been shown to induce cytokine response via TLR4 signaling, which leads to optimal maturation of APCs and their migration to the DLNs and activation of antigen-specific T and B cells. Based on these studies and our data, we speculate that EBOV GP possesses adjuvant-like properties.

Development of a safe and effective vaccine is important to prevent and combat EBOV infection and two virally vectored EBOV vaccine candidates, rVSV-ZEBOV and adenovirus-based vaccines have proceeded to clinical trials in multiple countries, including some with EVD endemic areas. In addition, EBOV VLPs containing GP were successful in protecting rodents and non-human primates from EBOV infection. Our recombinant EBOV GP possesses a proper trimeric conformation that resembles the GP present on the surface of Ebola virus particles, and most likely also on virally vectored vaccines. We surmise that this is the reason why recombinant EBOV GP is capable of inducing protective immune responses against EBOV. The current study, which is an extension of our previous study, further demonstrates that EBOV GP alone triggers fast, robust, yet balanced innate responses that may play a critical role in the induction of adaptive immunity. The strong early inflammatory responses observed in EVD survivors and rapid protection in individuals who received the rVSV-ZEBOV vaccine highlight the importance of well-regulated innate immune responses in post-exposure protection against EBOV infection. Post-exposure vaccination has been shown to prevent several viral diseases such as rabies, hepatitis B, and small pox in humans. There is an urgent need to develop effective post-exposure treatments in response to future filovirus outbreaks, to combat bioterrorism, or to treat laboratory exposures. Recent studies reported that VLPs protect mice from EBOV infection when given 24 h post-challenge. VSV-based vaccines given at high dosage levels have also been successfully used in the post-exposure prophylaxis in animals. However, undesirable reactogenic responses observed in significant numbers of rVSV-ZEBOV vaccinated human subjects when used at high doses raises some concerns with the use of this vaccine in special populations that may also be at risk of acquiring EVD. Our previous and current studies together demonstrated that recombinant EBOV GP can induce appropriate, but confined inflammatory responses and therefore shows a clear safety advantage over virally vectored vaccines. In summary, our data provides in vivo mechanistic evidence that recombinant EBOV GP triggers proper innate activation in the absence of adjuvants, which may lead to protection in naïve individuals against EBOV infection. Additionally, the knowledge gained from this study aids in a better understanding of the immunogenicity of EBOV GP and lays a foundation to test its potential use in pre- or post-exposure prophylaxis and for Ebola vaccine development.

### EXAMPLE 2

We studied how Ebola virus glycoprotein enhances immune responses by activation of macrophages and dendritic cells.

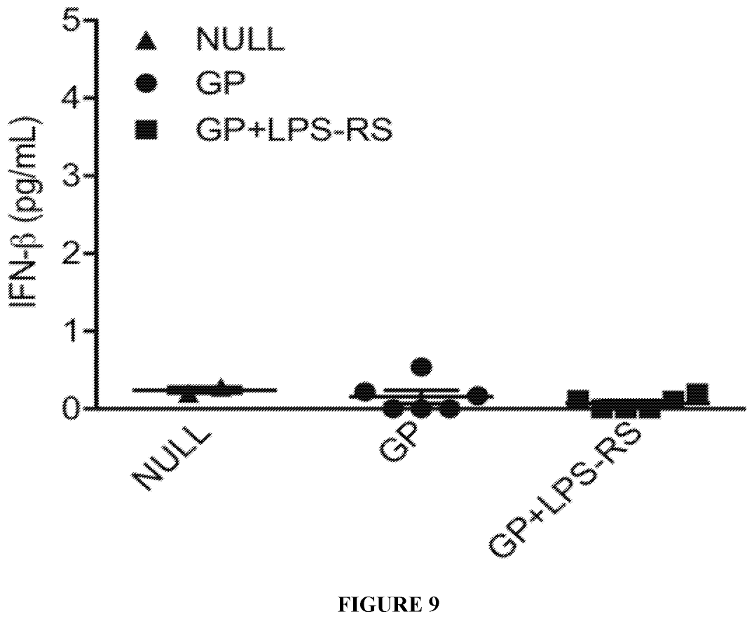

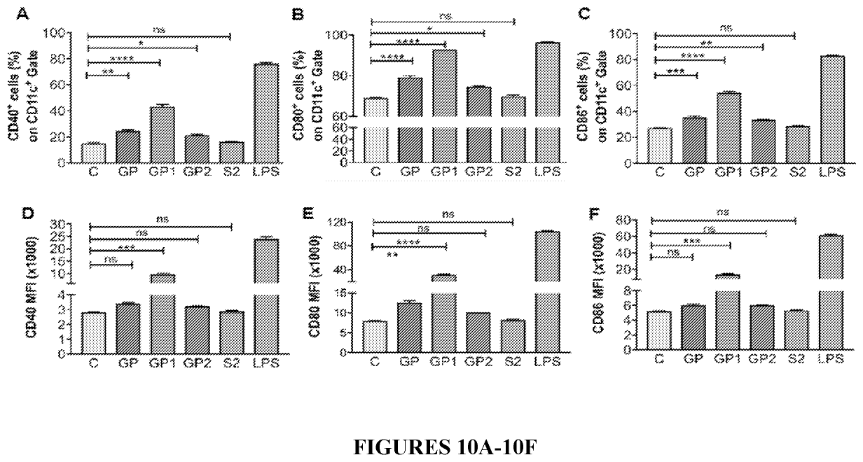

Ebola virus (EBOV) structural glycoprotein (GP) is composed of GP1 and GP2 subunits which form a heterodimer that is connected by a single disulfide bond between GP1 and GP2 subunits. We have generated recombinant GP protein using the Drosophila S2 expression system and demonstrated purified recombinant GP elicits a robust innate immune response in the absence of adjuvants or other viral components both in vitro and in vivo. Antigen-presenting cells (APCs), dendritic cells (DCs) and macrophages, are central for both activation of innate immune responses and initiation of adaptive immunity. To further elucidate which subunit mostly contributes to the innate responses induced by GP, we tested the ability of EBOV GP subunits (separated by size-exclusion chromatography after reducing the disulfide bond) to trigger activation of APCs. Exposure of EBOV GP led to elevated expression of costimulatory molecules CD40, CD80, CD86 on the surface of mouse bone marrow-derived DCs (BMDCs), which is essential for the priming of T cell responses. Moreover, treatment of mouse BMDCs with EBOV GP1 induced significantly higher expression of costimulatory molecules, indicating that GP1 has a stronger immunostimulatory effect (FIG. 10). Similarly, GP1 subunit induces higher levels of transcription of key inflammatory cytokine genes (TNF-α and IL-1β) in bone marrow-derived macrophages (BMDMs) of C57BL/6 mice within 2 hours of exposure (FIG. 11). Overall, EBOV GP, specifically GP1, is an effective stimulator of APCs and has potentials in enhancing innate and adaptive immune responses.

### EXAMPLE 3

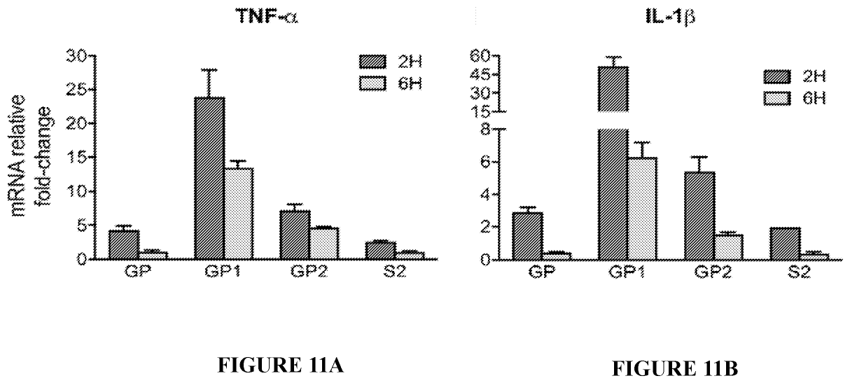

Analysis of the glycosylation status of the GP antigen was conducted using enzymatic deglycosylation with analysis on protein gels. For GP, the PNGase treatment resulted in a protein which migrated faster on SDS-PAGE, consistent with the removal of the carbohydrate side chains from all N-linked glycosylation sites (FIG. 12). In contrast, no evidence was found for 0-linked glycosylation using the EDEGLY kit. Reduction of the GP protein results in separation of GP1 and GP2 fragments (FIG. 12) and confirms that the furin cleavage site is being processed completely during post-translational processing.

### EXAMPLE 4

This example describes expression and purification of filovirus subunit proteins.

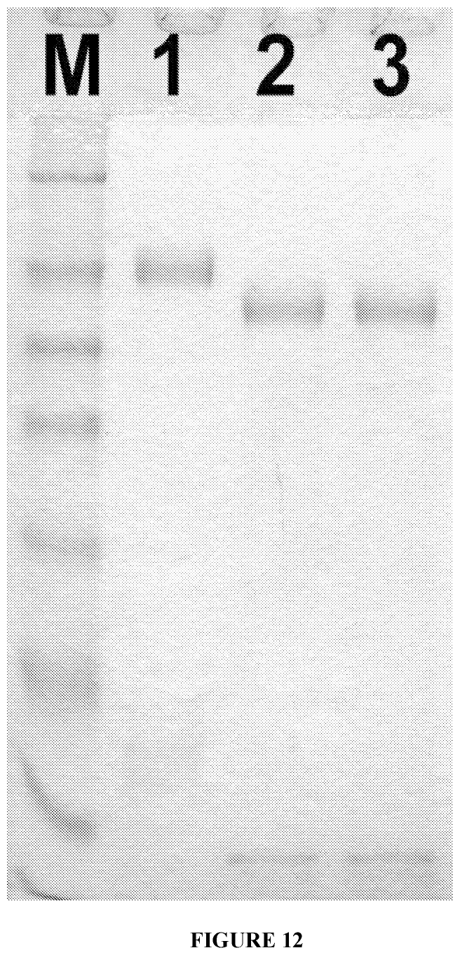

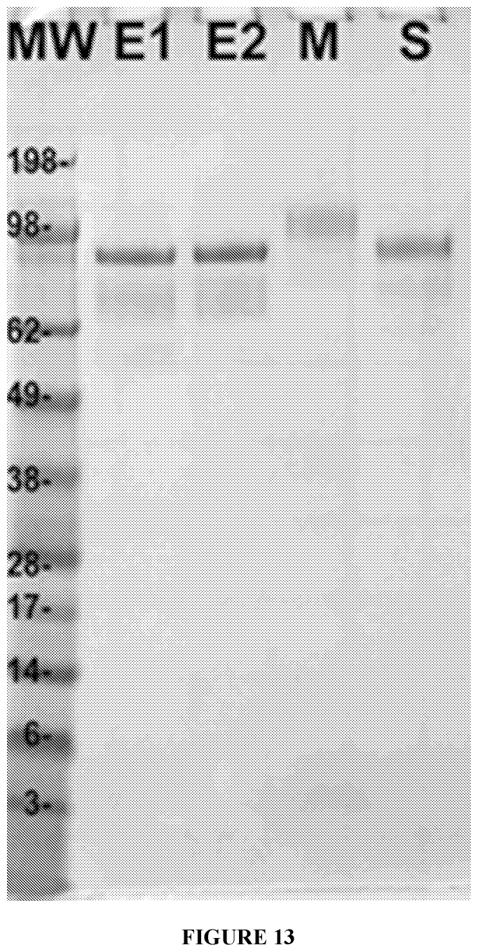

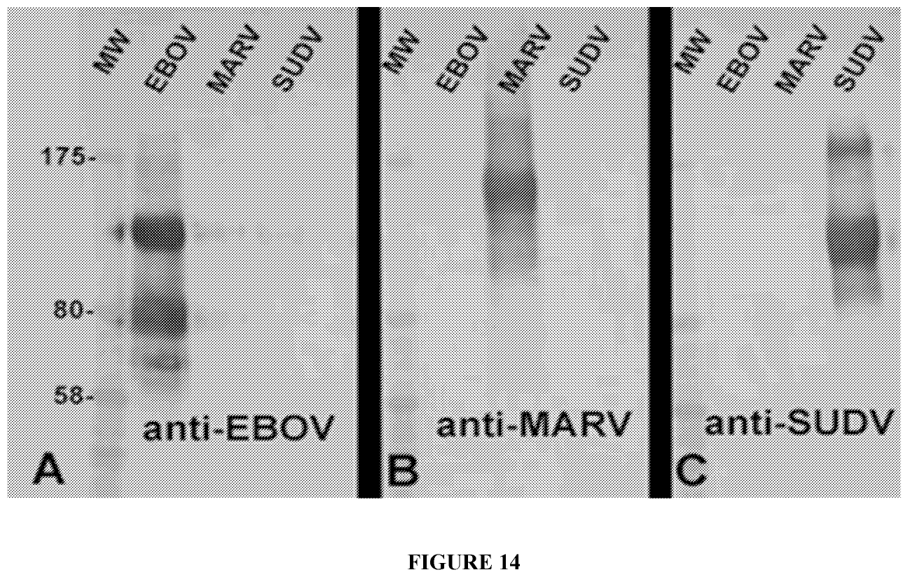

Filovirus antigens used for the studies described here have been expressed using stably transformed Drosophila cell lines in 1-5 L batches in a WAVE bioreactor (GE Lifesciences, Piscataway, N.J.). Expression levels of all selected cell lines (for MARV-GP after two rounds of subcloning) have been stable in the range of 5-10 mg/L. The GP subunits were subsequently purified by single-step immunoaffinity chromatography (IAC) using specific affinity columns for each individual protein (FIG. 13). To date, we have produced more than 200 mg of EBOV GP (E-GP), 50 mg of MARV GP, and 20 mg of SUDV GP with purity levels between 90-95% (based on SDS-PAGE). EBOV, SUDV and MARV GP's are highly pure and show good antigenic specificity (see a panel of Western blots containing all three GP's in FIG. 14). To establish the equivalency of plant- and murine hybridoma-derived monoclonal antibodies we tested plant- and hybridoma-derived anti-GP antibody (13C6): A 1.5 ml column containing 15 mg of immobilized plant-expressed antibody bound 0.2 mg antigen per batch while a column using hybridoma-derived antibody (100 mg immobilized on 10 ml NHS-sepharose) bound between 1-1.3 mg per batch proving that the plant-expressed antibody achieves similar yields and purity (>90%) of E-GP. We now routinely use plant-expressed monoclonal antibodies for production of filovirus GPs. When analyzing the size of purified E-GP, we discovered that it mainly forms trimers (native conformation on virus particles) as well as dimers of trimers (see FIG. 15). We separated the two populations of oligomers by FPLC and established their protective potential in guinea pigs as identical (data not shown). Therefore, a polishing step is not required in establishing our final antigen purification procedure.

### EXAMPLE 5

This example describes immunogenicity and efficacy studies performed in mice.

Immunogenicity of purified EBOV GP subunits was tested in Balb/c mice. First, individual antigens were tested in formulations with four functionally different adjuvants: ISA-51 (water-in-oil emulsion; Seppic, Fairfield, N.J.), GPI-0100 (saponin-based; Hawaii Biotech, Inc., Honolulu, Hi.), CoVaccine HT (emulsion-based; BTG, London, UK) and Ribi R-700 (monophosphoryl lipid A and trehalose dicorynomycolate; Sigma-Aldrich). Excellent humoral and cell-mediated responses were seen, especially for CoVaccine HT and GPI-0100 (data not shown). ELISA antibody responses to the antigens were evident after one immunization, and as expected, increased following a booster injection. E-GP administered at doses from 1-9 μg showed a typical dose-related response (data not shown). Balb/c mice were immunized at days 0, 28 and 56 with formulations containing IAC purified recombinant E-GP with or without adjuvants. The animals were infected 23 days after the third immunization by i.p. injection with 100 pfu (3000 LD50) of mouse adapted EBOV (MA-EBOV). The results of the experiment are shown in Table 1.

TABLE 1

| | | | Survival (day 20 | |
|---|---|---|---|---|
| Group No. | Immunogen^{[a]} | Adjuvant | post challenge)^{[b]} | Morbidity^{[c]} |
| 1 | GP | NONE | 70% | All survivors sick |
| 2 | GP | GPI-0100 | 90% | All survivors sick |
| 3 | GP | CoVaccine HT | 100% | None sick |
| 4 | NONE | NONE | 0% | No survivors |
| 5 | NONE | GPI-0100 | 10% | Survivor sick |
| 6 | NONE | CoVaccine HT | 0% | No survivors |

^{[a]}Mice were immunized with 10 μg antigen (s.c.)
^{[b]}10 animals per group, except groups 4 and 6 with 9 animals each.
^{[c]}Morbid (sick) animals showed any signs of illness (e.g. ruffled fur).

Interestingly, animals immunized three times with 10 μg of EBOV GP (no adjuvant) showed 70% protection, similar to protection reported after four doses of the best adjuvanted formulations of recombinant “Ebola immune complexes” and also similar in protection achieved with four doses of a recombinant GP-Fc fusion protein administered to mice in Freund's adjuvant, but significantly better than protective efficacy of Novavax's GP nanoparticles when given alone or in combination with Alum. GP formulated with CoVaccine HT showed 100% protective efficacy against both morbidity and mortality emphasizing the importance of adjuvant selection for protection. The excellent protective efficacy of the adjuvanted formulations, in combination with the finding of surprisingly good protective efficacy with unadjuvanted GP, strongly support the use of this protein as a vaccine candidate. In comparison, with and without adjuvant, recombinant GP yields immune responses equivalent or superior to responses seen with Ebola virus-like particles (VLPs) in mice, without the production challenges associated with VLPs that are being produced similarly to viruses using centrifugation methods and are prone to be affected by batch-to-batch consistency and stability issues.

### EXAMPLE 6

Well-characterized (“GMP-like”) recombinant antigens can be expressed by the Drosophila expression system at pilot scale and the antigens can be purified efficiently and economically by immunoaffinity chromatography (IAC) using antibodies produced in plants (“plantibodies”).

Pilot lot production utilizing bench scale, full process methods: In an effort to aid in the transfer of lab scale process to cGMP manufacturing, HBI has recently developed a bench scale full-process (GMP-like) process for a West Nile virus recombinant envelope protein which includes viral clearance steps. This represents a scaled down version of a cGMP process previously using single-use technology (GE Healthcare-Xcellerex). This scaled down process operates in a closed system (controlled environment and direct fluid transfers) including sterile filtration directly into bioprocess bags, including the final filtration step of the bulk product. All containers, tubing and fluid contact surfaces are sterile. No microbial or endotoxin contamination has been detected in any of the lots produced using this bench scale full process procedure. As an example of the comparability of this bench scale full-process to cGMP, the results of a WN-80E 200 L cGMP production compared to WN-80E bench scale production are presented in Table 4. The bench scale process represents an approximate scale down of 1/8th. This bench scale full-process is transferable to the production of filovirus GP proteins as the process has been specifically refined over the years for recombinant proteins produced in S2 cells and purified using IAC methods. Successful application of these methods to GP proteins greatly aids in the transfer of these proteins to cGMP manufacturing.

TABLE 4
Comparison of Full Scale GMP Process to Bench Scale Process

| | Scale | | | |
|---|---|---|---|---|
| Process Step | GMP | | Bench-Scale | |
| Cell Culture | 200 | L | 25 | L |
| IAC column volume | 1.3 | L | 150 | mL |
| Total volume purified | 187 | L | 24 | L |
| Total mg processed | 2806.5 | mg | 432 | mg |
| No. of IAC cycle/volume | 5/37 | L | 3/8 | L |
| WN-80E eluate pool volume | 45 | L | 3.6 | L |
| Viral inactivation starting volume | 45 | L | 3.6 | L |
| Viral filtration starting volume | 46 | L | 3.7 | L |
| UF/DF starting volume | 55 | L | 3.9 | L |
| UF/DF end volume | 1.5 | L | 269 | mL |
| Conc. of WN-80E | 1 | mg/mL | 0.8 | mg/mL |
| Yield of WN-80E | 1500 | mg | 215 | mg |

| | | |
|---|---|---|
| Overall efficiency of process | 53% | 49% |

The required research cell banks for GP antigens are successfully generated and verified. Sufficient quantities of the required plantibodies needed for protein purification are generated. Pilot lots for the GP antigens are produced utilizing bench scale full process methods that incorporate the plantibodies. These pilot lots for each of the three GP antigens demonstrate successful pilot production methods from 25 L cultures capable of yielding at least 100 mg purified antigen.

Lyophilization of antigens can be performed as follows. Vaccine formulation used in preliminary data section: 0.1 mg/mL EBOV GP, 10 mM ammonium acetate pH 7, 9.5% (w/v) trehalose ±0.5 mg/mL aluminum hydroxide. Lyophilizer shelves were pre-cooled to -10° C.; shelf temperature was decreased at a rate of 0.5° C./min to −40° C. and then held at-40° C. for 1 hour. Primary drying at 60 mTorr and −20° C. for 20 hours. Secondary drying at 60 mTorr with temperature gradient to 0° C., then 30° C. followed by hold at 30° C. for 5 hours.

### EXAMPLE 7

This example describes studies that explore non-human primate (NHP) immunogenicity and efficacy. Cynomolgus macaques (Macaca fascicularis) constitute the most susceptible EBOV challenge model used for pivotal vaccine efficacy studies. We recently completed NHP efficacy testing.

Macaques were immunized by the intramuscular route (IM) three times at 3-week intervals with 25 μg of EBOV GP formulated with 10 mg of CoVaccine HT adjuvant while the control group was given only adjuvant. Four weeks after the last vaccination, all animals were challenged by the subcutaneous route (SC) with 1000 LD50 of EBOV, strain Kikwit. While 2/2 controls died on day 6 after infection, 5/6 vaccines were completely protected. The single animal that met the euthanasia criteria in the vaccine group did not show any signs of Ebola Virus Disease (EVD) (based on clinical chemistry and the necropsy report). Viremia as determined by plaque assay on sera collected from all animals at 3-4 day intervals until death or day 28 (survivors) demonstrated that all vaccines, including the one animal that had to be euthanized, did not show any sign of systemic viremia suggesting complete protection against EVD (data not shown). Antibody titers determined on serum samples from vaccinated animals at various time points post vaccination and after challenge also demonstrated a robust humoral immune response (data not shown).

Collectively the results of the NHP efficacy study demonstrated full vaccine protection against live EBOV challenge, successful inhibition of viremia, and high antibody titers following vaccination.

### EXAMPLE 8

This example describes generation of recombinant structural subunits of EBOV glycoprotein. Different structural domains of EBOV GP can be expressed and purified from insect cell cultures as separate recombinant subunits.

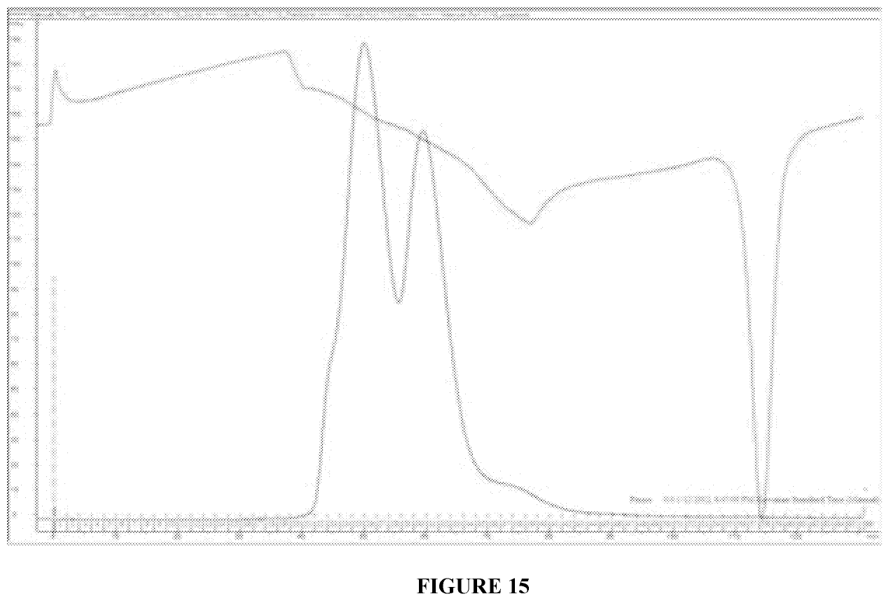

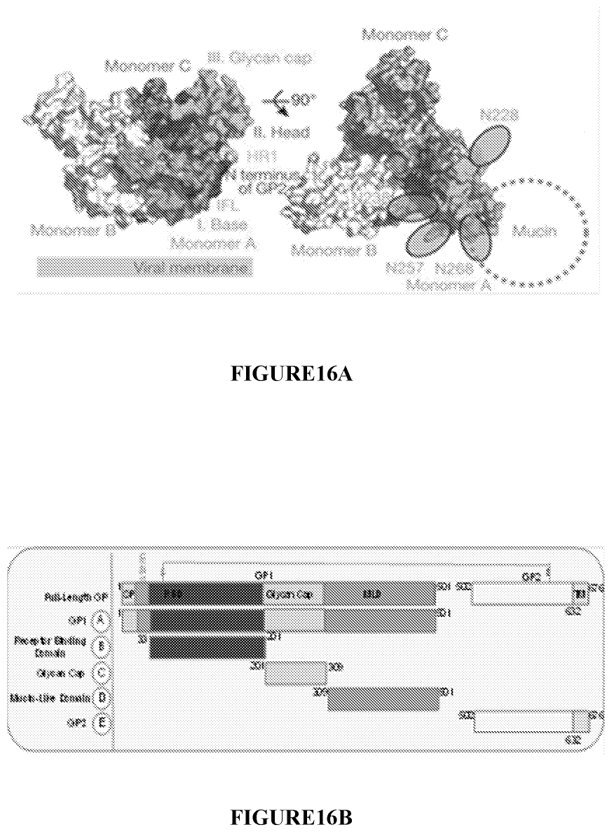

We have shown that high quality filovirus antigens at high yields can be generated using the Drosophila S2 expression system. The recombinant EBOV GP protects rodents and NHP against EBOV challenge. Additional experiments have demonstrated that these subunit proteins show innate immunostimulatory effects in vitro and in vivo. Dr. Saphire (TSRI) and colleagues have established a significant knowledge base regarding the structure of trimeric filovirus GP's and identified the relevant structural domains using most of the available monoclonal antibodies to fully map the 3D structure as well as quaternary interactions between domains. Expressing the structural domains (GP1, GP2, Receptor-binding domain (RBD), Glycan Cap (GC), and Mucin-Like domain (MLD) of EBOV GP (FIG. 16A) separately results in properly folded subunits without the risk of carrying over portions of other domains and therefore allows detailed analysis of their immunostimulatory effects.

EBOV GP1 and GP2 as well as subunits constituting the MLD, RBD, and GC as seen in a schematic in FIG. 16B are expressed and purified using domain-specific immunoaffinity-chromatography methods yielding structurally relevant subunit proteins.

Construction of EBOV GP subunit protein expression plasmids is as follows. Expression plasmids are generated to express the selected structural domains based on the amino acid sequence of the GP originated from the EBOV Mayinga (prototype) strain. Subunits are expressed as secreted proteins using the Drosophila BiP secretion signal which results in efficient processing after cleavage by cellular signalase during expression, releasing the subunits with native N-terminal structure and no further modifications into the supernatant. We have extensively demonstrated in prior work that N-linked glycosylations of viral glycoproteins (including EBOV GP) are uniformly processed using this approach. The expression plasmids are constructed by inserting PCR fragments generated from a cDNA plasmid containing the full length EBOV GP sequence into the pMTBiP expression vectors (Invitrogen, Carlsbad, Calif.).

Transformation and Culturing of Drosophila Cells are as follows. Drosophila melanogaster S2 cells are maintained in ExCell 420 serum free medium (MilliporeSigma). Transformation of S2 cells is accomplished utilizing Lipofectamine to co-transfect the cells with expression vectors and selectable marker plasmid (pCoHygro, Invitrogen). Transformants are selected by growth in ExCell 420 medium containing 300 μg/ml hygromycin B. For larger scale culturing of the Drosophila S2 cells for production of EBOV GP subunits, a Wave bioreactor (GE Healthcare, Piscataway, N.J.) is used.

Analysis of expression is as follows. Preliminary expression analysis of the GP subunit proteins is carried out in 5 ml cultures. 2×10⁶ cells/ml is induced with 200 μM CuSO₄ and grown for 7 days at 26° C. Cultures are evaluated for target proteins in cell lysates and culture supernatants using SDS-PAGE. Gels are either stained with Coomassie blue or electroblotted onto nitrocellulose. Domain-specific monoclonal antibodies are used to probe Western blots for expression and reactivity of the subunits. Recombinant proteins are easily detected in S2 culture medium by Coomassie staining of gels at expression levels above 1 mg/L.

TABLE 5
Monoclonal antibodies for immunoafinity purification of GP subunits

| | | | | Use for |
|---|---|---|---|---|
| | | | | purification of GP |
| Antibody | Epitope | Epitope Type | Specificity | subunit protein |
| KZ52 | GP1: 42-43 | Conformational | Gp1 (stem)-Gp2 | A, E |
| | GP2: 505-514: | | | |
| | 549-555 | | | |
| 13C6-1-1 | Epitope shared | Conformational | Gp1 | Used for GP |
| | with sGp and Gp | | sGp | purification, |
| | | | | A, B, C |
| 13F5-1-2 | GP1: 404-413 | Linear | GP1- Mucin-like | A, D |
| | | | Domain | |
| c1H3 | Overlaps with | Conformational | GP core: | A, B, C |
| | 13C6 | non-neutralizing | glycan cap | |
| c4G7 | GP1: 459-500 | Bind quaternary | GP1.2 | A, D |
| | | epitope on the GP | | |
| | | trimer | | |
| c2G4 | Overlaps with | Binds quarternary | GP2 | E |
| | c4G7 and KZ52 | epitope on the GP | | |
| | | trimer | | |

Purification is as follows. The subunits A-E (as identified in FIG. 16B) are purified from the culture supernatant by protein-specific immunoaffinity chromatography. We first select the strongest binders based on reactivity in western blots from the roster of monoclonal antibodies shown in table 5 above. Purified monoclonal antibodies for screening and purification purposes are obtained from Mapp Biopharmaceutical. The purified antibodies are then coupled individually to 1 ml HiTrap columns containing NETS-activated Sepharose (GE Healthcare).

Each of the structural subunits is expressed at satisfactory levels by stable transformant S2 cell lines. Suitable monoclonal antibodies can be selected from the available roster allowing the production of 10-20 mg of each subunit.

### EXAMPLE 9

This example explores how filovirus GP induces innate immunity in human and mouse immune cells. Filovirus GP activates multiple innate immune signaling pathways in a cell-type- and domain-specific manner.

We believe that to ultimately understand how GP induces protection, the first step is characterizing the early events of innate immunity, as they are the main determinants of protection and shaping the robustness of adaptive immunity. Although our recent study demonstrates that TLR4 is one of the PRR pathways activated by GP, it is likely that GP activates multiple PRR pathways and the cross talk and synergy between them play an important role in downstream events including fine-tuning of the innate-adaptive interface. There are several examples supporting that virus-derived proteins can act as a ligand for PRRs that are not typically associated with virus infections such as TLR3/7 and either positively or negatively regulate innate immune responses. For example, dengue virus NS1 has been shown to activate immune cells via TLR4. Also, the core protein of HCV can act as a ligand for gC1qR, a complement receptor for C1q and suppress production of IL-12 and Th1 immunity because of cross talk with TLR4. In addition, other studies reported that MARV and EBOV activate TREM-1 signaling, another positive regulator of inflammation in myeloid derived cells resulting in secretion of proinflammatory cytokines. These studies and our new data as described below suggest that activation of TLR4 by GP may lead to a better understanding of how different innate immune pathways collectively mediate a robust inflammatory response to GP antigen.

Our data collectively demonstrates that (a) GP alone can induce specific antibodies and partial protection against EBOV challenge in mice; (b) EBOV GP is efficiently endocytosed by mouse antigen presenting cells (APCs); (c) GP induces a robust inflammatory response in mice; (d) the GP-induced inflammatory response is mediated by the TLR4 pathway and affects homing of immune cells in the lymph nodes. These results strongly suggest that the signaling events induced by GP via TLR4 have implications beyond inducible innate responses and may include modulation of cell-mediated immunity. We similarly also characterized the role of innate immune and inflammatory pathways in immunity to West Nile virus (WNV) and Zika virus using mouse models and human immune cells.

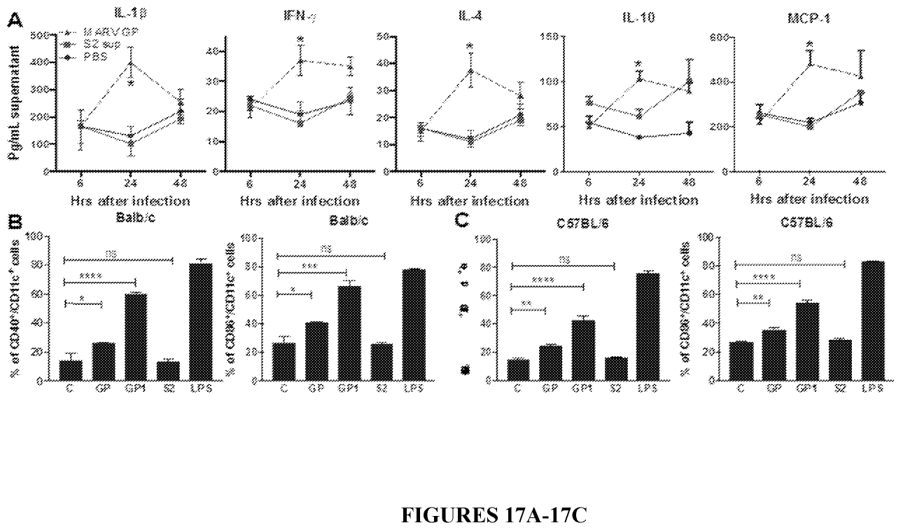

MARV GP can induce innate immune responses. We tested whether MARV GP exhibits similar pro-inflammatory responses as EBOV GP. In vivo assessment of the immune response showed that MARV GP also induced increased production of both key Th1 and Th2 cytokines and chemokines at 24 hrs after treatment in mice (FIG. 17, panel A).

CD40 and CD80 expression is induced by EBOV GP. As seen in FIG. 17, panels B and C, treatment with GP and especially GP1 (produced from full-length GP treated with denaturing and reducing agents and separated using size exclusion chromatography) increases expression of the co-stimulatory molecules CD40 and CD80 on B cells from both BALB/c and C57BL/6 mice.

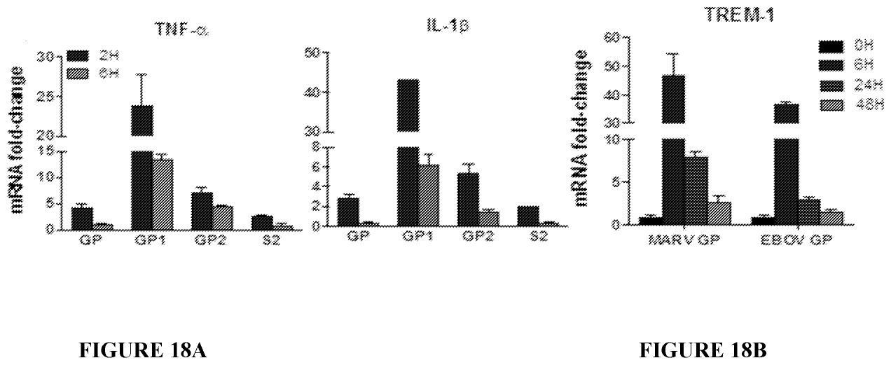

GP1 alone can induce cytokine responses. BMDMs from BALB/c and C57BL/6 mice were treated with 1 μg of GP1 (prepared from EBOV GP as described above) and full-length GP. GP1 induced significant expression of TNF-α and IL-1β transcripts and induced phenotypic maturation of BMDMs (FIG. 18, panel A).

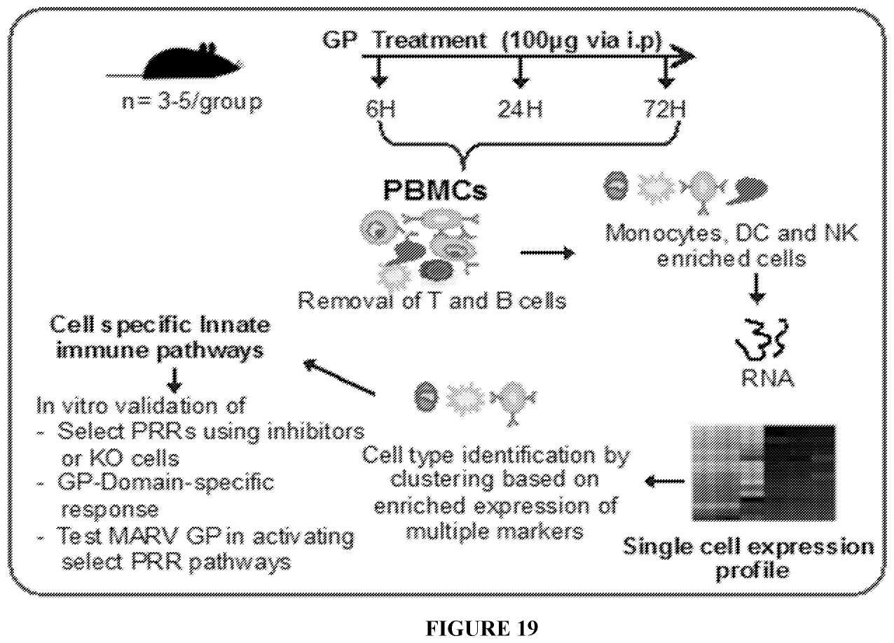

TREM-1 expression is induced by EBOV GP. In line with a report of TREM-1 induction by EBOV, we tested if EBOV GP alone can induce TREM-1 expression. As seen in FIG. 18, panel B, TREM-1 transcripts were significantly induced in human monocytes derived THP-1 cells by 25-fold in just 2 hours following GP treatment and remained high until 24 hours after treatment suggesting a key role of TREM-1 signaling in GP-associated inflammatory response. Characterization of cell- and domain-specific innate immune response to filovirus GP is performed as described in FIG. 19.

Animal experiments and cell types to focus are as follows. We believe that using an in vivo approach is more appropriate to characterize global transcriptome changes in the key immune cells in the blood and represents the changes induced by GP in a multicellular environment as compared to in vitro treatment of BMDMs and BMDCs. Although our data show that monocytes/macrophages and DCs are primary APCs responding to GP, recent studies also indicate that treatment of mouse NK cells with EBOV VLPs containing GP and VP40 resulted in enhanced cytokine secretion and can mediate protection in mice against EBOV challenge. Therefore we focus on monocytes, DC and NK cells in this experiment.

Male and female C57BL/6 mice (6-8 weeks old) are injected with 100 μg of EBOV GP or PBS alone via i.p route and at 6, 24 and 72 hrs after treatment, and whole blood is harvested. PBMCs are separated by lysing red blood cells followed by density gradient centrifugation. We remove T cells and B cells by positive selection using magnetic bead based kits from Miltenyi to obtain untouched monocytes, DC and NK enriched leukocytes. We target at least 300-1000 live cells of each cell type per mouse for each time point/sample for scRNA-Seq. Since the percentage of monocytes, DC and NK cells in the mouse blood is in the range of 1-5%, from 1-2 mL blood per mouse via cardiac puncture we obtain enough enriched leukocytes to freeze at least 1000-2000 cells of each cell type per mouse to cryofreeze for RNA-seq.

The monocytes, DC and NK cells are identified based on enriched expression of multiple known markers of these cells and analyzed to characterize enriched expression of genes. Based on the pathway enrichment p-values (Fisher's exact test) and activation z-scores, the highest activated networks are identified and a hierarchical clustering heatmap showing a list of significant canonical pathways and functional processes of biological importance induced by GP is generated.

Comparison of the innate immune response to GP and different structural units in vitro is as follows. BMDMs pooled from 3-8 BALB/c and C57BL/6 mice each are treated with at least two dose levels (5 and 10 m) of GP, equimolar concentrations of subunits A-E and controls (two doses of VSV-EBOV-MOI 1 and 10). Key innate immune cytokines are measured using the multiplex Luminex assay. This experiment allows us to compare and identify the specific regions of the GP most associated with innate immune responses.

Validation of the association of key innate immune pathways induced by GP with cytokine production and antigen presentation response using mouse and human immune cells is as follows. RNA-Seq analysis identifies at least 2-4 innate immune pathways activated by GP in addition to TLR4. Mouse BMDMs and BMDCs from BALB/c and C57BL/6 mice and human blood-derived macrophages are treated with GP, GP1 and up to 2 select truncations in the presence/absence of antagonist or inhibitors of TLR4, TREM-1 and two select PRRs. In some cases, if an inhibitor for the select PRR pathway is not available, we consider conducting studies using BMDMs and BMDCs from mice deficient in those PRRs. At 2, 6, 12 and 24 hrs after treatment, we measure the levels of multiple cytokines in the media using the Luminex assay. Finally we also test whether innate immunity induced by GP via these select PRRs pathways can affect maturation of APCs. The expression of costimulatory activation markers CD40, CD80 (B-7.1), CD86 (B-7.2), MHC class I and II molecules in CD11C^{hi} and CD11b populations are measured in GP treated mouse BMDMs and BMDCs at different time points using flow cytometry. Subsequently, the same strategy is applied to MARV GP as it is genetically the most distant member of human pathogenic filoviruses. We treat these immune cells with GP from Marburg filovirus in the presence of PRR inhibitors and measure the cytokine production in the media. To further test if GP-induced innate immune signaling pathways are dependent on the direct association of GP with select PRR receptors, we use an immunoprecipitation assay to determine the direct binding of GP with the cell surface receptors. Total protein extracted from mouse BMDM/DC at early time points (1, 6, 12 and 24 hours) after GP treatment is used to precipitate GP with magnetic beads bound to our antibody used for purification and then immunoblotted for TLR4, TREM-1 and other select receptors using commercially available antibodies. If needed, we also conduct reverse assays to precipitate individual receptors and then immunoblot with anti-GP antibody.

Relative changes in multiple innate immune pathways including direct interaction with receptors and downstream adaptor proteins in response to GP treatment are identified. Exemplary activations are of TLR4, TREM-1, some complement receptors and other positive or negative innate immune modulators.

### EXAMPLE 10

This example explores evaluation of the protective efficacy of GP-induced innate immunity in a mouse challenge model dependent and independent of adaptive immunity. Robust induction of innate immunity via the TLR4 and other immune signaling pathways following administration of GP directly improves antiviral response and causes enhanced protection against lethal challenge with various filoviruses.

Participation of innate immunity induced by GP in protection against lethal filovirus challenge in mouse models is assessed. We first evaluate how the cell-mediated and antibody responses regulated by GP via TLR4 affect the disease outcome in mice to extend the mouse efficacy previously studied, and further investigate two other PRR pathways. Using the virally-vectored rVSV-ZEBOV vaccine candidate as a control allows us to evaluate which of these pathways are preferentially triggered by the GP protein and which may be activated by other components of the VSV vector. We further investigate the effect of GP in pre- and post-exposure treatment.

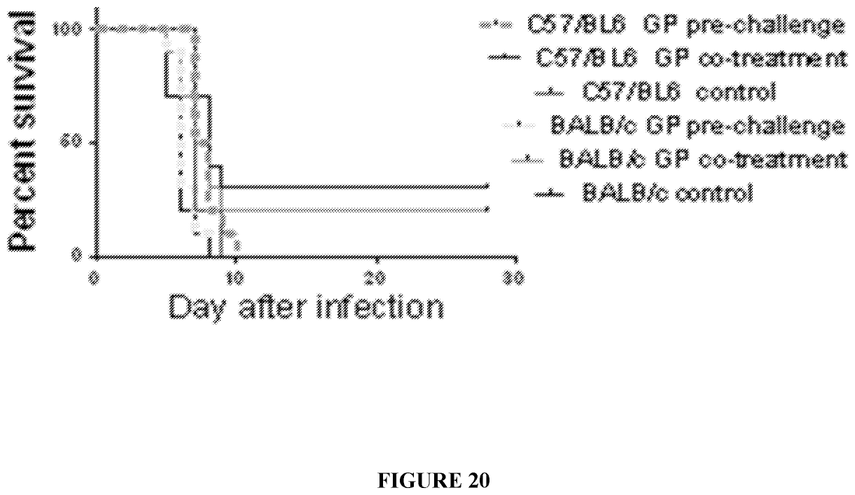

We conducted a mouse study to determine suitable mouse strains and treatment schedules. Groups of ten naïve BALB/c or C57BL/6 mice each were either pre-treated with 100μg GP at 24 h prior to or co-treated with the same dose of GP at the time of challenge with 1000 PFU of mouse-adapted EBOV (maEBOV). Antigen and virus were administered via the i.p route. Survival in comparison to untreated control animals (FIG. 20) shows that while pre-treatment did not improve survival in this uniformly lethal challenge experiment, co-treatment protected 2/10 BALB/c and 3/10 C57BL/6. The survival curves were significantly different from control groups (p<0.05 using the Gehan-Breslow-Wilcoxon test). In addition, analysis of cytokines in sera of infected animals revealed that there are significant differences in the IFN-γ response comparing co-treated survivors and controls in both strains of mice by day 4, with the same trends evident at day 1 (data not shown). This shows that both mouse models can be used to further investigate the effect of innate immune mechanisms on EBOV infection. Optimization of the treatment schedule may further improve the effect on protection against EBOV infection.

BALB/c or C57BL/6 mice are treated with GP, GP1 and at least one additional GP truncation variant, as well as rVSV-ZEBOV in the presence or absence of specific PRR inhibitors to test the effect of various immune signaling pathways on the generation of protective immunity. We test TLR4 and up to two additional pathways implicated in innate immune activation by GP via the use of pathway-specific inhibitors or suitable knockout mouse models. After confirming additional immune signaling pathways and the structural features of GP involved in their induction, we evaluate their effect on short-term protection against EBOV and MARV challenge independent of adaptive immunity both in wild-type and selected knockout mouse models.

In the first experiment we assess the role that TLR4-mediated GP immunity plays in generating protective antibodies. Groups of ten BALB/c mice (of both sexes), each vaccinated three times with either 10 μg of EBOV GP, 7.39 μg of GP1, or an equimolar amount of the selected GP subunit alone in the absence or presence of the TLR4 antagonist, LPS-RS (administered at −48 h, −24 hr and concurrent with each GP immunization), is challenged with 1000 PFU (30,000 LD50) of mouse adapted EBOV. Controls include a protective GP-based formulation (with 1mg CoVaccine HT) and a virally vectored vaccine (rVSV-ZEBOV) administered at 2×10E4 PFU with or without the TLR4-antagonist. Mice are bled pre-challenge to document immunogenicity and at 24 and 120 hrs after infection to test for cytokines (by multiplex assay) and viremia (by RT-PCR). This experiment is repeated two more times (Exp 1B/1C) to test two additional PRR pathways. If suitable antagonists of these PRR's are not available, groups 4-6 and 9 (Table 6) can use PRR-knockout mouse models congenic with the other groups. Therefore, this study may have to be conducted in C57BL/6 mice.

TABLE 6
Mouse challenge experiment 1

| | Vaccine | Adjuvant or | |
|---|---|---|---|
| Exp 1 | Antigen | Antagonist | Purpose |
| 1 | EBOV GP | NONE | Full-length GP alone |
| 2 | EBOV GP1 | NONE | GP1 alone |
| 3 | EBOV GP subunit | NONE | GP truncation alone |
| 4 | EBOV GP | LPS-RS | GP + TLR4 antagonist |
| 5 | EBOV GP1 | LPS-RS | GP1 + TLR4 antagonist |
| 6 | EBOV GP subunit | LPS-RS | GP truncation + TLR4 antagonist |
| 7 | EBOV GP | CoVaccine HT | Positive control |
| 8 | VSV-ZEBOV | NONE | Virally vectored positive control |
| 9 | VSV-ZEBOV | LPS-RS | Test importance of TLR4 pathway |
| | | | for VSV vaccine |
| 10 | NONE | NONE | Negative control |

Mouse challenge experiment 2 is as follows. Guided by our data (FIG. 20), we assess the effect of GP in concurrent and post-exposure treatment and examine antigen dosing. Groups of ten mice are treated with EBOV GP alone (at 200, 100 or 50 μg, IP) at two time points concurrent with and 24 hr after challenge with 1000 PFU of mouse adapted EBOV (Table 7). Sera collected at 24 and 120 hrs after infection are tested for cytokines and viremia. The lowest successful dose (>50% protection) is used in subsequent experiments. In experiment 2B, this dose of GP and an equal molar amount of a GP subunit that was shown to activate the TLR4 pathway is tested as described for experiment 2A (Table 8). *dose level as selected in experiment 2A **truncation variant selected based on mouse experiment 1A (GPI or other GP subunit tested) and dose level calculated to match molarity of full length GP.

TABLE 7
Mouse challenge experiment 2A -
groups of 10 BALB/c mice are used

| | | Treatment |
|---|---|---|
| Exp 2A | Antigen | timing |
| 1 | 50 μg EBOV GP | With challenge |
| 2 | 50 μg EBOV GP | +24 h |
| 3 | 100 μg EBOV GP | With challenge |
| 4 | 100 μg EBOV GP | +24 h |
| 5 | 200 μg EBOV GP | With challenge |
| 6 | 200 μg EBOV GP | +24 h |
| 7 | NONE | NONE |

TABLE 8
Mouse challenge experiment 2B -10 BALB/c mice per group

| Exp 2B | Antigen | Treatment timing |
|---|---|---|
| 1 | X μg EBOV GP * | With challenge |
| 2 | X μg EBOV GP * | +24 h |
| 3 | Y μg GP subunit ** | With challenge |
| 4 | Y μg GP subunit ** | +24 h |
| 5 | NONE | NONE |

Mouse challenge experiment 3 is as follows. To determine the impact of the TLR4 pathway on the protective innate immunity generated by EBOV GP in vivo, groups of ten mice are treated with GP at the ideal dose level selected in mouse challenge experiment 2A with and without LPS-RS at two time points, concurrent with and 24 hr after challenge with 1000 PFU of mouse adapted EBOV as depicted in table 9. Mice are bled at 24 and 120 hrs after infection to test for cytokines and viremia, and monitored for mortality Experiment 3B further confirms the pathway by using TLR4 knockout mice in a C57BL/6 background (Table 10).

Mouse challenge experiment 4 is as follows. The ability of a second PRR signaling pathway to modulate the protective innate immunity generated by EBOV GP administration is tested. Mouse experiment 4A is identical in layout to experiment 3A and investigates an additional innate immune pathway and confirmed in a mouse challenge experiment 1B or 1C using an available PRR antagonist. Mouse experiment 4B utilizes a corresponding PRR knockout mouse model and is similar in design to experiment 3B.

TABLE 9
Mouse challenge experiment 3A - groups
of 10 C57/BL6 mice are used

| | | Antagonist | Treatment |
|---|---|---|---|
| Exp 3A | Antigen | dosing | timing |
| 1 | X μg EBOV GP * | NONE | With challenge |
| 2 | X μg EBOV GP * | NONE | +24 h |
| 3 | Y μg GP subunit ** | NONE | With challenge |
| 4 | Y μg GP subunit ** | NONE | +24 h |
| 5 | NONE | NONE | NONE |
| 6 | X μg EBOV GP * | −48 h, −24 h, 0 h | With challenge |
| 7 | X μg EBOV GP * | −48 h, −24 h, 0 h | +24 h |
| 8 | Y μg GP subunit ** | −48 h, −24 h, 0 h | With challenge |
| 9 | Y μg GP subunit ** | −48 h, −24 h, 0 h | +24 h |
| 10 | NONE | −48 h, −24 h, 0 h | NONE |

TABLE 10
Mouse challenge experiment 3B -10 TLR4−/−
mice (C57/BL6 background) per group

| | | Antagonist | Treatment |
|---|---|---|---|
| Exp 3B | Antigen | dosing | timing |
| 1 | X μg EBOV GP * | NONE | With challenge |
| 2 | X μg EBOV GP * | NONE | +24 h |
| 3 | Y μg GP subunit ** | NONE | With challenge |
| 4 | Y μg GP subunit ** | NONE | +24 h |
| 5 | NONE | NONE | NONE |

Mouse challenge experiment 5 is as follows. To address the question whether the protective innate immune effect of a filovirus GP is also observed using MARV GP, similar to experiment 2A, groups of ten mice each is treated with MARV GP or EBOV GP at the three dose levels, using concurrent and 24hr post-challenge treatments in conjunction with viral challenge using 1000 PFU of mouse adapted MARV (Table 11). Mice are bled at 24 and 120 hrs after infection to test for cytokines and viremia.

TABLE 11
Mouse challenge experiment 5 - groups
of 10 BALB/c mice are used

| Exp 5 | Antigen | Treatment timing |
|---|---|---|
| 1 | 50 μg MARV GP | With challenge |
| 2 | 50 μg MARV GP | +24 h |
| 3 | 100 μg MARV GP | With challenge |
| 4 | 100 μg MARV GP | +24 h |
| 5 | 200 μg MARV GP | With challenge |
| 6 | 200 μg MARV GP | +24 h |
| 7 | 50 μg EBOV GP | With challenge |
| 8 | 50 μg EBOV GP | +24 h |
| 9 | 100 μg EBOV GP | With challenge |
| 10 | 100 μg EBOV GP | +24 h |
| 11 | 200 μg EBOV GP | With challenge |
| 12 | 200 μg EBOV GP | +24 h |
| 13 | NONE | NONE |

In mouse challenge experiment 1, a result is full protection in the positive control groups, no survival in the negative control groups and between 50-80% protection in animals treated with GP alone, while animals also receiving the PRR antagonist show a lower (0-20%) protection. Survival is correlated with the level of GP-specific IgG titers in serum samples and also with markedly increased post-challenge cytokine levels (such as IL-4 and IFN-y). Mouse challenge experiment 2 demonstrates partial protection (at least 30%) against EBOV infection, particularly in animals treated with GP concurrent with or after challenge and allows us to determine the optimal dose of GP for subsequent experiments and confirm the domain-specific effect of a selected subunit. Mouse challenge experiment 3A verifies the previous results in C57BL/6 mice (groups 1-5) and expands results by also co-treating the animals with the TLR4 antagonist LPS-RS, which eliminates or reduces the protection engendered by GP-induced innate immunity. The effect of TLR4 is further validated by using a knockout mouse model in experiment 3B. The subsequent mouse challenge experiment tests the effect of an additional PRR pathway analogous to the prior experiment and demonstrates the effect of inhibitors and knockout on challenge outcome. The final challenge experiment demonstrates that the effect observed in the context of EBOV infection can be extended to other filoviruses by testing MARV GP and EBOV GP treatments in the MARV mouse challenge model.

### EXAMPLE 11

This example explores the elucidation of the mechanisms by which filovirus GP-induced innate immunity modulates antibody responses. GP-induced innate activation activates germinal center (GC) B cell and T follicular helper (Tfh) cell responses that subsequently lead to the production of antigen-specific antibodies.

The germinal center is a highly specialized microenvironment within secondary lymphoid organs where antigen-specific B cells receive help from cognate CD4+T follicular helper (Tfh) cells and undergo selection and expansion, affinity maturation, and class switching. GC reaction is required for T helper cell-dependent antibody production, which is critical for the humoral responses to pathogens, and has been used to evaluate vaccine efficacy. Recent vaccine development strategies including influenza virus vaccine have focused on promoting a potent Tfh response, which directly controls the magnitude of GC B cell reaction. A recent EBOV vaccine study using virus-like particles (VLPs) in mice also demonstrated that protective humoral responses are generated through Tfh cell-dependent GC reactions. However, from prior studies it remains unclear which components and mechanisms contribute to the production of vaccine-induced GC responses. In murine models, the activation of APCs, specifically the enhanced expression of co-stimulatory molecules (CD80, CD86) in DCs, has been associated with the generation of antigen-specific follicular helper T cells, GC B cells, and high-affinity class-switched antibody production. Additionally, stimulation of innate immune PRRs such as TLRs enhances the GC response. Although our recent study provides evidence for the involvement of the TLR4 pathway in GP-associated innate immunity and upregulation of co-stimulatory molecules on APCs by GP and GP1 (FIG. 17, panels B/C), the link between these responses and the development of antibody responses, specifically the GC reaction, remains unknown. Therefore, we first investigate whether EBOV GP or GP subunits/domains induce a GC response and then identify specific PRR pathways involved in modulating the GC response development and subsequent antibody production.

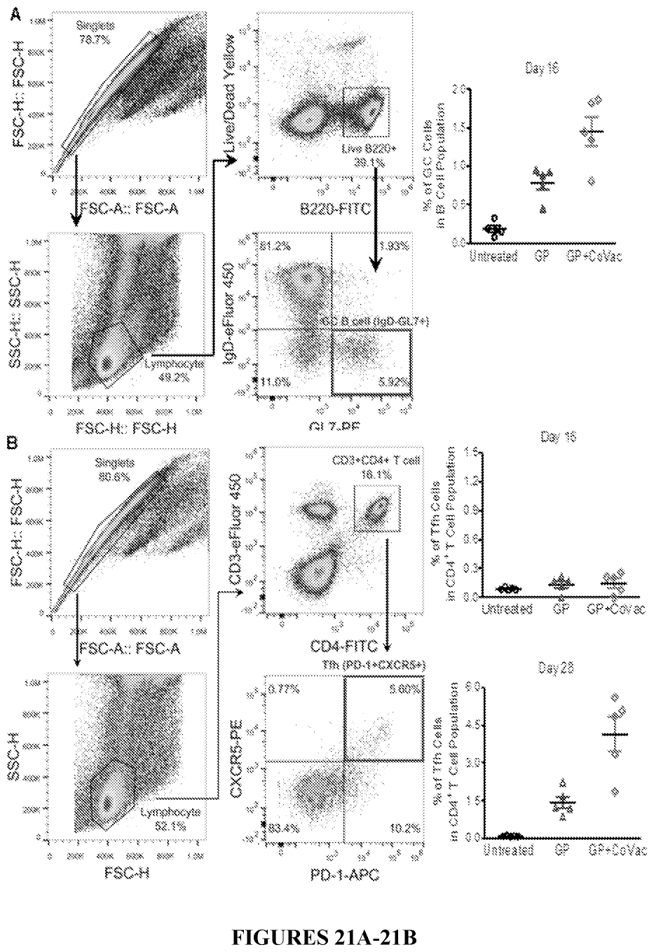

Our experiments examined the GC responses induced by GP in the presence or absence of adjuvant in vivo. CoVaccine HT is the lead adjuvant used in the formulation of our recombinant subunit vaccine and has been shown to enhance the vaccine efficacy in mice and NHPs. BALB/c mice (n=5) were administered 2 doses of 10 μg EBOV GP or 10 μg GP formulated with 1 mg CoVaccine HT intraperitoneally (i.p.) at 3-week intervals. Splenic GC responses were analyzed by measuring the frequencies of GC B cells and Tfh cells using flow cytometry at two weeks post 1st dose (Day 16) and one week post 2nd dose (Day 28). The results showed that (i) GP treatment enhanced the frequencies of GC B cells and Tfh cells both in the absence or presence of CoVaccine HT (FIG. 21). This suggests that it is possible to further elucidate the GC responses elicited by individual GP subunits/domains. (ii) In the presence of CoVaccine HT we observed a higher proportion of mouse splenic GC B cells and Tfh cells, suggesting that an adjuvant may be required to enhance GC responses if only one or two doses are administered. Therefore, in order to examine GP or GP subunit-induced GC responses, we immunize mice with 3 doses of antigens with or without CoVaccine HT. As the presence of CoVaccine HT enhances the frequencies of GC B cells and Tfh cells significantly, comparing GC responses between GP and GP subunit-immunized groups is informative. Our previous in vitro and in vivo data have demonstrated there is no significant difference in GP-induced innate immune responses between C57BL/6 and BALB/c mice. Therefore, we use BALB/c mice to determine whether GP or specific GP domains elicit GC responses and produce the most diverse B cell repertoire, and use C57BL/6 mice to validate role of select PRRs in modulating GC responses due to availability of knock out mice on this background.

To define the GC responses induced by EBOV GP and GP subunits/domains, mouse experiments and characterization of GC reactions are as follows. Groups of ten 8- to 10-week-old BALB/c mice are immunized with the same molar concentration of purified full-length (FL) GP and the 5 truncations with and without CoVaccine HT (Table 8—i.m. at week 0, i.m.at W3, i.m. at W6, and Spleen/LN/Serum at W7) via the intramuscular (i.m) route followed by 2 booster doses at 3-week intervals. Single dose immunization of mice with rVSV-ZEBOV (2×10E4 PFU) serves as a positive control and no antigen groups with or without adjuvant as negative controls. At day 7 after the 3rd immunization, sera are collected and draining lymph nodes (DLNs) and spleens are harvested for the subsequent analysis. GP-specific total IgG, IgG1, IgG2a, IgG2b and IgG3 antibodies are measured in the serum from different groups of GP-treated and control mice using FL-GP as antigen by our in-house developed microsphere-based IgG immunoassay.

TABLE 12

| Group | Antigens | Adjuvant |
|---|---|---|
| 1 | EBOV GP-FL | None |
| 2 | EBOV GP1 | None |
| 3 | EBOV GP2 | None |
| 4 | EBOV GP1-RBD | None |
| 5 | EBOV GP1-Glycan cao | None |
| 6 | EBOV GP1-Mucin like domain | None |
| 7 | EBOV GP-FL | CoVaccine HT |
| 8 | EBOV GP1 | CoVaccine HT |
| 9 | EBOV GP2 | CoVaccine HT |
| 10 | EBOV GP1-RBD | CoVaccine HT |
| 11 | EBOV GP1-Glycan cao | CoVaccine HT |
| 12 | EBOV GP1-Mucin like domain | CoVaccine HT |
| 13 | VSV-ZEBOV | None |
| 14 | None | CoVaccine HT |
| 15 | None | None |

Analysis of GC B cells and Tfh is as follows. Single cell suspensions prepared from DLNs and spleens are stained for specific markers by fluorochrome-conjugated antibodies: B220, IgD, CD95, GL-7, CD3, CD4, CXCR5, PD-1. The frequencies of GC B cells and Tfh cells in the DLNs and spleens are analyzed using flow cytometry as described in FIG. 21. GC B cells are defined as IgD-GL7+CD95+ cells in B220+ B cell populations. Tfh cells are defined as B220-PD1+CXCR5+CD4+ T cells.

Characterization of GC B cell repertoires is as follows. Highly purified GC B cells (B220+GL7+CD95+) in DLNs or spleens of mice immunized with GP and select GP subunits implicated in eliciting strong GC responses are isolated by cell sorting on a BD FacsAria. DNA extraction and Next-Generation sequencing (NGS) of murine B cell receptor (BCR) heavy chain VDJ loci can be performed by Seattle Genomics Institute. The BCR sequences are obtained from amplicons spanning the framework region 3 of the Ig heavy chain variable (IGHV)-gene segment to the 3′ end of the complete VDJ junction. The Ig properties, including IGHV family usage, somatic hypermutation (SHM) percentage, and genetic characteristics of the BCR complementarity-determining region are analyzed and compared between GP and GP subunit groups with or without CoVaccine HT.

Measurable titers of various IgG subtypes in both GP-treated mice with and without CoVaccine HT are a result. In the presence of CoVaccine HT, robust frequencies of splenic or lymphoid GC B cells and Tfh cells are a result in mice immunized with FL-GP and select GP subunits such as GP1. Relatively higher frequencies of GC B cells and Tfh cells in mice immunized with the same GP subunit in the absence of adjuvant are also a result. The clonality of GC B cell repertoires in both GP and GP subunit groups increases relative to the naive B cell repertoire in the unvaccinated control group.

To determine whether the innate signaling affects the development of GP-induced humoral responses, we examine whether TLR4, TREM-1 and up to two select PRRs are involved in mediating GP-induced GC responses. Our data indicates that GP1 induces a more robust innate immune response than full-length GP (FIG. 18, panel A).

Animal experiments are as follows. C57BL/6 WT or congenic mice deficient in the selected PRR pathways are administered 10 μg of GP or the same molar concentration of GP subunits (Table 13) via the i.m. route. At day 7 after the third immunization, GP-specific antibodies and the frequencies of GC B cells and Tfh cells are compared in each group of WT and KO mice.

TABLE 13

| | Antigens (with |
|---|---|
| Groups | and without |
| (n = 10) | CoVaccineHT) |
| WT | EBOV GP-FL |
| WT | EBOV GP1 |
| WT | EBOV GP subunit |
| TLR4 KO | EBOV GP-FL |
| TLR4 KO | EBOV GP1 |
| TLR4 KO | EBOV GP subunit |
| PRR-X KO | EBOV GP-FL |
| PRR-X KO | EBOV GP1 |
| PRR-X KO | EBOV GP subunit |
| PRR-Y KO | EBOV GP-FL |
| PRR-Y KO | EBOV GP1 |
| PRR-Y KO | EBOV GP subunit |
| WT | None |
| TLR4 KO | None |
| PRR-X KO | None |
| PRR-Y KO | None |

Mice deficient in TLR4 and other PRRs exhibit lower frequencies of GC B cells and Tfh cells as compared to WT mice, suggesting that GP-activated innate immune pathways contribute to the GC reaction.

Quantitative data from virus burden, Luminex, and flow cytometry assays is analyzed using 2-way ANOVA or Mann-Whitney test to compare values between various groups. The survival data is summarized by Kaplan-Meier curves and compared by a log-rank test. Results are considered significant at p<0.05. The animal numbers are increased if required to achieve statistical significance. In vitro experiments are conducted at least in triplicates, and for animal experiments gender as a biological variable is considered.

The data on how full length and specific regions of GP activate innate immunity, fine-tune adaptive immunity and directly mediate the short term innate-immunity based protection has a significant impact on further development of filovirus vaccines and fills a critical gap in the elucidation of filovirus vaccine mechanisms of protection.

Although the invention has been described with reference to the above examples, it will be understood that modifications and variations are encompassed within the spirit and scope of the invention. Accordingly, the invention is limited only by the following claims.

What is claimed is:

1. A composition comprising:
an isolated glycosylated polypeptide comprising at least about 20 amino acid residues of an envelope glycoprotein of a filovirus, wherein the glycosylated polypeptide corresponds to one or more structural subunits of the glycoprotein; and
a pharmaceutically acceptable carrier.

2. The composition of claim 1, wherein administration of the composition to a subject stimulates an innate immune response in the subject.

3. The composition of claim 2, wherein the administration of the composition is in absence of an adjuvant.

4. The composition of claim 3, wherein the administration of the composition to the subject stimulates the innate immune response via a TLR4 pathway.

5. The composition of claim 3, wherein the administration of the composition to the subject stimulates production of one or more cytokines selected from the group consisting of IL-1beta, IL-2, IL-4, IL-5, IL-6, IL-10, IFN-gamma, TNF-alpha, and combinations thereof.

6. The composition of claim 1, wherein the glycosylated polypeptide comprises at least 50 amino acid residues of the surface glycoprotein of a filovirus selected from the group consisting of a Marburg marburgvirus (MARV) and a Sudan ebolavirus (SUDV).

7. The composition of claim 1, wherein the glycosylated polypeptide comprises at least 50 amino acid residues of the envelope glycoprotein of a Zaire ebolavirus (EBOV).

8. The composition of claim 7, wherein the glycosylated polypeptide comprises an amino acid sequence that is at least 95% identical to an amino acid sequence range selected from the group consisting of 1-501, 33-501, 33-201, 201-309, 309-501, and 502-676 of SEQ ID NO: 2.

9. The composition of claim 7, wherein the glycosylated polypeptide comprises an amino acid sequence that is at least 95% identical to amino acid sequence range 33-501 of SEQ ID NO: 2.

10. The composition of claim 7, wherein the glycosylated polypeptide comprises at least one N-linked glycosylation.

11. The composition of claim 1, further comprising one or more additional isolated glycosylated polypeptides each of which comprises at least 50 amino acid residues of the surface glycoprotein of a filovirus, wherein each of the additional glycosylated polypeptides corresponds to one or more structural subunits of the glycoprotein.

12. The composition of claim 11, wherein the composition is a multivalent vaccine.

13. A method of inducing an innate immune response in a subject comprising administering an effective amount of the composition of claim 1 to the subject, thereby inducing an innate immune response.

14. The method of claim 13, wherein the effective amount of the composition is administered to the subject in absence of an adjuvant.

15. The method of claim 13, wherein the induced innate immune response comprises production of cytokines via a TLR4 pathway.

16. The method of claim 13, wherein the induced innate immune response enhances expression of costimulatory molecules CD40, CD80, and CD86 on the surface of bone marrow-derived dendritic cells.

17. A method of producing the composition of claim 1 comprising expressing an antigen comprising the glycosylated polypeptide in Drosophila S2 cells and isolating the polypeptide.

18. The method of claim 17, further comprising purifying the glycosylated polypeptide using single-step immunoaffinity chromatography (IAC).

19. The method of claim 18, wherein the IAC comprises an affinity column having a monoclonal antibody.

20. The method of claim 18, wherein the purified glycosylated polypeptide has a three-dimensional structure that differs from the corresponding one or more structural subunits of the glycoprotein by less than 10 Angstroms in root-mean-square deviation of C alpha atomic coordinates after optimal rigid body superposition.


SEQUENCE LISTING (2 sequences)

SEQ ID NO: 1 - DNA, 2406 bp, Ebola virus
gatgaagattaagccgacagtgagcgtaatcttcatctctcttagattatttgttttccagagtaggggtcgtcaggtccttttcaatcgtgtaaccaaaataaactccactagaaggatattgtggggcaacaacacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttgacatcaatctagttatctctttgagaatgataaacttgatgaagattaagaaaaa

SEQ ID NO: 2 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF